SEQUENCE LISTING (76 sequences)

SEQ ID NO: 1 - protein, 156 aa, Burkholderia cepacia
MPLLSASIVSAPVVTSETYVDIPGLYLDVAKAGIRDGKLQVILNVPTPYATGNNFPGIYFAIATNQGVVADGCFTYSSKVPESTGRMPFTLVATIDVGSGVTFVKGQWKSVRGSAMHIDSYASLSAIWGTAAPSSQGSGNQGAETGGTGAGNIGGG

SEQ ID NO: 2 - protein, 4 aa, synthetic construct
  REGION 1..4: note = Synthesized peptide
RRRR

SEQ ID NO: 3 - protein, 5 aa, synthetic construct
  REGION 1..5: note = Synthesized peptide
RRRRR

SEQ ID NO: 4 - protein, 6 aa, synthetic construct
  REGION 1..6: note = Synthesized peptide
RRRRRR

SEQ ID NO: 5 - protein, 7 aa, synthetic construct
  REGION 1..7: note = Synthesized peptide
RRRRRRR

SEQ ID NO: 6 - protein, 10 aa, synthetic construct
  REGION 1..10: note = Synthesized peptide
RRRRRRRRRR

SEQ ID NO: 7 - protein, 79 aa, Homo sapiens
SSVLASCPKKPVSSYLRFSKEQLPIFKAQNPDAKTTELIRRIAQRWRELPDSKKKIYQDAYRAEWQVYKEEISRFKEQL

SEQ ID NO: 8 - DNA, 65 bp, synthetic construct
  misc_feature 1..65: note = Synthesized oligonucleotide
  source 1..65: mol_type = other DNA
gatattccgggtctgtatctcgatgttgcgaaagcgggcatacgcgatgggaaactgcaggtgat

SEQ ID NO: 9 - DNA, 62 bp, synthetic construct
  misc_feature 1..62: note = Synthesized oligonucleotide
  source 1..62: mol_type = other DNA
cgacgctcttccgatctctgatgttgcgaaagcgggcatacggtcagatcggaagagcacac

SEQ ID NO: 10 - protein, 143 aa, Agaricus bisporus
MTYTISIRVYQTTPKGFFRPVERTNWKYANGGTWDEVRGEYVLTMGGSGTSGSLRFVSSDTDESFVATFGVHNYKRWCDIVTNLTNEQTALVINQEYYGVPIRDQARENQLTSYNVANAKGRRFAIEYTVTEGDNLKANLIIG

SEQ ID NO: 11 - DNA, 78 bp, synthetic construct
  misc_feature 1..78: note = Synthesized oligonucleotide
  source 1..78: mol_type = other DNA
tgggatgaagtgcgtggagagtacgttctgactatgggtggctctgggacctcaggaagcttacggttcgtttcgagt

SEQ ID NO: 12 - DNA, 62 bp, synthetic construct
  misc_feature 1..62: note = Synthesized oligonucleotide
  source 1..62: mol_type = other DNA
cgacgctcttccgatctctgtgggtggctctgggacctcagggtcagatcggaagagcacac

SEQ ID NO: 13 - protein, 149 aa, Laetiporus sulphureus
MTDIYIPPEGLYFRLLGFASRQVIFARNSPSPDVGLSPVNDQATDQYFSLIYGTGEHAGLYAIKSKATGKVLFSRRPAEPYVGQIDGDGRYPDNWFKIEPGKTYLSKYFRLVQPSTGTALVSRTHLQPYFWNHPQTEVFDDQYFTFLFE

SEQ ID NO: 14 - DNA, 77 bp, synthetic construct
  misc_feature 1..77: note = Synthesized oligonucleotide
  source 1..77: mol_type = other DNA
cgacatctacattccgccggaaggcttgtactttcgcttgcttgggtttgccagtcgccaggtgattttcgcacgga

SEQ ID NO: 15 - DNA, 63 bp, synthetic construct
  misc_feature 1..63: note = Synthesized oligonucleotide
  source 1..63: mol_type = other DNA
cgacgctcttccgatctctgcgcttgcttgggtttgccagtcggtcagatcggaagagcacac

SEQ ID NO: 16 - DNA, 94 bp, synthetic construct
  misc_feature 1..94: note = Synthesized oligonucleotide
  source 1..94: mol_type = other DNA
tgcatcccgtaccatcctgttgctacagaacaatatctatgccgctagccagggttggactgtgtcaaataatgtacagcccatcgttgcttcg

SEQ ID NO: 17 - DNA, 62 bp, synthetic construct
  misc_feature 1..62: note = Synthesized oligonucleotide
  source 1..62: mol_type = other DNA
cgacgctcttccgatctctgtgccgctagccagggttggactgtcagatcggaagagcacac

SEQ ID NO: 18 - DNA, 66 bp, synthetic construct
  misc_feature 1..66: note = Synthesized oligonucleotide
  source 1..66: mol_type = other DNA
tcggcaatgctgtaagtgacttaatcctccaaggagccgccacaccggacaaggggaccgtcaaac

SEQ ID NO: 19 - DNA, 62 bp, synthetic construct
  misc_feature 1..62: note = Synthesized oligonucleotide
  source 1..62: mol_type = other DNA
cgacgctcttccgatctctgtcctccaaggagccgccacaccgtcagatcggaagagcacac

SEQ ID NO: 20 - protein, 313 aa, Aleuria aurantia
MPTEFLYTSKIAAISWAATGGRQQRVYFQDLNGKIREAQRGGDNPWTGGSSQNVIGEAKLFSPLAAVTWKSAQGIQIRVYCVNKDNILSEFVYDGSKWITGQLGSVGVKVGSNSKLAALQWGGSESAPPNIRVYYQKSNGSGSSIHEYVWSGKWTAGASFGSTVPGTGIGATAIGPGRLRIYYQATDNKIREHCWDSNSWYVGGFSASASAGVSIAAISWGSTPNIRVYWQKGREELYEAAYGGSWNTPGQIKDASRPTPSLPDTFIAANSSGNIDISVFFQASGVSLQQWQWISGKGWSIGAVVPTGTPAGW

SEQ ID NO: 21 - DNA, 86 bp, synthetic construct
  misc_feature 1..86: note = Synthesized oligonucleotide
  source 1..86: mol_type = other DNA
cgcgattagctggggttcaaccccgaacattcgcgtttactggcagaaaggtcgcgaagagctgtacgaagccgcttatggtggtt

SEQ ID NO: 22 - DNA, 64 bp, synthetic construct
  misc_feature 1..64: note = Synthesized oligonucleotide
  source 1..64: mol_type = other DNA
cgacgctcttccgatctctgactggcagaaaggtcgcgaagagcgtcagatcggaagagcacac

SEQ ID NO: 23 - DNA, 99 bp, synthetic construct
  misc_feature 1..99: note = Synthesized oligonucleotide
  source 1..99: mol_type = other DNA
gtgacgccacaacaggaacagatggtaacttggaactcacaagggtgtcaagtaatgggagtccacagggaagcagtgtgggccgggctttgttctatg

SEQ ID NO: 24 - DNA, 64 bp, synthetic construct
  misc_feature 1..64: note = Synthesized oligonucleotide
  source 1..64: mol_type = other DNA
cgacgctcttccgatctctgtgggagtccacagggaagcagtgtgtcagatcggaagagcacac

SEQ ID NO: 25 - DNA, 76 bp, synthetic construct
  misc_feature 1..76: note = Synthesized oligonucleotide
  source 1..76: mol_type = other DNA
atttcctcgatttggcaagcaagtggtctggggtcaactccgtggcgtagcgaaatctttgattgcgatgacttcg

SEQ ID NO: 26 - DNA, 64 bp, synthetic construct
  misc_feature 1..64: note = Synthesized oligonucleotide
  source 1..64: mol_type = other DNA
cgacgctcttccgatctctgggtctggggtcaactccgtggcgtgtcagatcggaagagcacac

SEQ ID NO: 27 - DNA, 74 bp, synthetic construct
  misc_feature 1..74: note = Synthesized oligonucleotide
  source 1..74: mol_type = other DNA
caatcagacgcgtattggtgtccaaccgcgcaacatcacgaccaaagggtttgactgcgtgttttatacgtgga

SEQ ID NO: 28 - DNA, 64 bp, synthetic construct
  misc_feature 1..64: note = Synthesized oligonucleotide
  source 1..64: mol_type = other DNA
cgacgctcttccgatctctgtccaaccgcgcaacatcacgaccagtcagatcggaagagcacac

SEQ ID NO: 29 - DNA, 73 bp, synthetic construct
  misc_feature 1..73: note = Synthesized oligonucleotide
  source 1..73: mol_type = other DNA
caggcaatagctgggaaaacgtctttagcggctggtgtgtaggggccaatacggctagtacccaaggtctgtc

SEQ ID NO: 30 - DNA, 62 bp, synthetic construct
  misc_feature 1..62: note = Synthesized oligonucleotide
  source 1..62: mol_type = other DNA
cgacgctcttccgatctctgagcggctggtgtgtaggggccagtcagatcggaagagcacac

SEQ ID NO: 31 - DNA, 75 bp, synthetic construct
  misc_feature 1..75: note = Synthesized oligonucleotide
  source 1..75: mol_type = other DNA
ttggtgatgccggtgtttacgtcgccctgaacaacggcaatggcacatttggcccggttaaacgcgtgatcgaca

SEQ ID NO: 32 - DNA, 64 bp, synthetic construct
  misc_feature 1..64: note = Synthesized oligonucleotide
  source 1..64: mol_type = other DNA
cgacgctcttccgatctctgtcgccctgaacaacggcaatggcagtcagatcggaagagcacac

SEQ ID NO: 33 - DNA, 68 bp, synthetic construct
  misc_feature 1..68: note = Synthesized oligonucleotide
  source 1..68: mol_type = other DNA
gcagaacgactttaaacccgaatctgtggcagccattcgttcctccgcgtttaacagcaaaggtggca

SEQ ID NO: 34 - DNA, 64 bp, synthetic construct
  misc_feature 1..64: note = Synthesized oligonucleotide
  source 1..64: mol_type = other DNA
cgacgctcttccgatctctgtgtggcagccattcgttcctccgcgtcagatcggaagagcacac

SEQ ID NO: 35 - DNA, 74 bp, synthetic construct
  misc_feature 1..74: note = Synthesized oligonucleotide
  source 1..74: mol_type = other DNA
atcgtgaacatcccgatcagggtctgatttgccacgatgcgttctgtggagccttggtgatgaagatcggcaat

SEQ ID NO: 36 - DNA, 64 bp, synthetic construct
  misc_feature 1..64: note = Synthesized oligonucleotide
  source 1..64: mol_type = other DNA
cgacgctcttccgatctctgtgccacgatgcgttctgtggagccgtcagatcggaagagcacac

SEQ ID NO: 37 - DNA, 75 bp, synthetic construct
  misc_feature 1..75: note = Synthesized oligonucleotide
  source 1..75: mol_type = other DNA
cgttggattgtgacgacacactcggatggtactcgcaccttggaaaccgtaggcatcaacagcagtgccttcata

SEQ ID NO: 38 - DNA, 64 bp, synthetic construct
  misc_feature 1..64: note = Synthesized oligonucleotide
  source 1..64: mol_type = other DNA
cgacgctcttccgatctctgtggtactcgcaccttggaaaccgtgtcagatcggaagagcacac

SEQ ID NO: 39 - DNA, 75 bp, synthetic construct
  misc_feature 1..75: note = Synthesized oligonucleotide
  source 1..75: mol_type = other DNA
tgcaaccagttggggtacggttccgtccattcgcgtctacaccgcgaacaacggcaagatcaccgaacgttgctg

SEQ ID NO: 40 - DNA, 64 bp, synthetic construct
  misc_feature 1..64: note = Synthesized oligonucleotide
  source 1..64: mol_type = other DNA
cgacgctcttccgatctctgtccgtccattcgcgtctacaccgcgtcagatcggaagagcacac

SEQ ID NO: 41 - DNA, 73 bp, synthetic construct
  misc_feature 1..73: note = Synthesized oligonucleotide
  source 1..73: mol_type = other DNA
gtgttcgcgatgacaatctcgtcgatggtgcagctctggttggctcacaacagcctactccggtttccattga

SEQ ID NO: 42 - DNA, 62 bp, synthetic construct
  misc_feature 1..62: note = Synthesized oligonucleotide
  source 1..62: mol_type = other DNA
cgacgctcttccgatctctgtggtgcagctctggttggctcagtcagatcggaagagcacac

SEQ ID NO: 43 - DNA, 75 bp, synthetic construct
  misc_feature 1..75: note = Synthesized oligonucleotide
  source 1..75: mol_type = other DNA
tctcctttgtcatcgatgcacctaacaaagccaaagctgcagatggccttgccttcttccttgcacctgtggata

SEQ ID NO: 44 - DNA, 64 bp, synthetic construct
  misc_feature 1..64: note = Synthesized oligonucleotide
  source 1..64: mol_type = other DNA
cgacgctcttccgatctctggccaaagctgcagatggccttgccgtcagatcggaagagcacac

SEQ ID NO: 45 - DNA, 71 bp, synthetic construct
  misc_feature 1..71: note = Synthesized oligonucleotide
  source 1..71: mol_type = other DNA
cgaattggccaagaacatcacctttgcgagtccgtattgccgtccaccggtagttctgctgagcattaccc

SEQ ID NO: 46 - DNA, 64 bp, synthetic construct
  misc_feature 1..64: note = Synthesized oligonucleotide
  source 1..64: mol_type = other DNA
cgacgctcttccgatctctggcgagtccgtattgccgtccaccggtcagatcggaagagcacac

SEQ ID NO: 47 - DNA, 70 bp, synthetic construct
  misc_feature 1..70: note = Synthesized oligonucleotide
  source 1..70: mol_type = other DNA
gcgataatgctaagtggcaggtagcactcgttgctgggtctggggacagcgccgagtacctgatcatcaa

SEQ ID NO: 48 - DNA, 64 bp, synthetic construct
  misc_feature 1..64: note = Synthesized oligonucleotide
  source 1..64: mol_type = other DNA
cgacgctcttccgatctctggcactcgttgctgggtctggggacgtcagatcggaagagcacac

SEQ ID NO: 49 - DNA, 75 bp, synthetic construct
  misc_feature 1..75: note = Synthesized oligonucleotide
  source 1..75: mol_type = other DNA
ctggtcaaaatcggtccatggggtggcaatggaggcagtgcacaagacatctcggttccacccaagaaactgctt

SEQ ID NO: 50 - DNA, 64 bp, synthetic construct
  misc_feature 1..64: note = Synthesized oligonucleotide
  source 1..64: mol_type = other DNA
cgacgctcttccgatctctggggtggcaatggaggcagtgcacagtcagatcggaagagcacac

SEQ ID NO: 51 - DNA, 71 bp, synthetic construct
  misc_feature 1..71: note = Synthesized oligonucleotide
  source 1..71: mol_type = other DNA
gaccatctactactcctcaaccatggtgaacggcctgcaagtcgtgtatggcaatgggacgacaaaactcc

SEQ ID NO: 52 - DNA, 64 bp, synthetic construct
  misc_feature 1..64: note = Synthesized oligonucleotide
  source 1..64: mol_type = other DNA
cgacgctcttccgatctctgtggtgaacggcctgcaagtcgtgtgtcagatcggaagagcacac

SEQ ID NO: 53 - DNA, 72 bp, synthetic construct
  misc_feature 1..72: note = Synthesized oligonucleotide
  source 1..72: mol_type = other DNA
tactcaaatccaagcgtccaggttggcgaagtctcaatcggcgatcggagtctgaattcgggtacaggtagc

SEQ ID NO: 54 - DNA, 64 bp, synthetic construct
  misc_feature 1..64: note = Synthesized oligonucleotide
  source 1..64: mol_type = other DNA
cgacgctcttccgatctctgggcgaagtctcaatcggcgatcgggtcagatcggaagagcacac

SEQ ID NO: 55 - DNA, 78 bp, synthetic construct
  misc_feature 1..78: note = Synthesized oligonucleotide
  source 1..78: mol_type = other DNA
cacctaatcccagtacagcagctactgatggtttagcgttcttcttggcacccgctgatacacaaccacaatctgccg

SEQ ID NO: 56 - DNA, 62 bp, synthetic construct
  misc_feature 1..62: note = Synthesized oligonucleotide
  source 1..62: mol_type = other DNA
cgacgctcttccgatctctgagcgttcttcttggcacccgctgtcagatcggaagagcacac

SEQ ID NO: 57 - DNA, 74 bp, synthetic construct
  misc_feature 1..74: note = Synthesized oligonucleotide
  source 1..74: mol_type = other DNA
caatatgggccgtcgttttgggccgtatggcggtagcggtggtagcgcgaatacgctgtcaaacgtcaaagtga

SEQ ID NO: 58 - DNA, 63 bp, synthetic construct
  misc_feature 1..63: note = Synthesized oligonucleotide
  source 1..63: mol_type = other DNA
cgacgctcttccgatctctgcgtatggcggtagcggtggtagcgtcagatcggaagagcacac

SEQ ID NO: 59 - DNA, 62 bp, synthetic construct
  misc_feature 1..62: note = Synthesized oligonucleotide
  source 1..62: mol_type = other DNA
acgattaccgacgatcaacacgtgctgacgatgggtggcagtggaacctctggtaccttacg

SEQ ID NO: 60 - DNA, 62 bp, synthetic construct
  misc_feature 1..62: note = Synthesized oligonucleotide
  source 1..62: mol_type = other DNA
cgacgctcttccgatctctgcgtgctgacgatgggtggcagtgtcagatcggaagagcacac

SEQ ID NO: 61 - DNA, 64 bp, synthetic construct
  misc_feature 1..64: note = Synthesized oligonucleotide
  source 1..64: mol_type = other DNA
cgacgctcttccgatctctgtggcaagtctgctgggatcatggcgtcagatcggaagagcacac

SEQ ID NO: 62 - DNA, 63 bp, synthetic construct
  misc_feature 1..63: note = Synthesized oligonucleotide
  source 1..63: mol_type = other DNA
cgacgctcttccgatctctggtgggaatggtggctcagcgttcgtcagatcggaagagcacac

SEQ ID NO: 63 - DNA, 62 bp, synthetic construct
  misc_feature 1..62: note = Synthesized oligonucleotide
  source 1..62: mol_type = other DNA
cgacgctcttccgatctctgactgatgcgcgtttagccccgagtcagatcggaagagcacac

SEQ ID NO: 64 - DNA, 64 bp, synthetic construct
  misc_feature 1..64: note = Synthesized oligonucleotide
  source 1..64: mol_type = other DNA
cgacgctcttccgatctctgacccaagcgatctgactgtccaccgtcagatcggaagagcacac

SEQ ID NO: 65 - DNA, 62 bp, synthetic construct
  misc_feature 1..62: note = Synthesized oligonucleotide
  source 1..62: mol_type = other DNA
cgacgctcttccgatctctgggcggcaacaatcccattgcgtgtcagatcggaagagcacac

SEQ ID NO: 66 - DNA, 63 bp, synthetic construct
  misc_feature 1..63: note = Synthesized oligonucleotide
  source 1..63: mol_type = other DNA
cgacgctcttccgatctctgtggcctttcacttcaacccacgcgtcagatcggaagagcacac

SEQ ID NO: 67 - DNA, 61 bp, synthetic construct
  misc_feature 1..61: note = Synthesized oligonucleotide
  source 1..61: mol_type = other DNA
cgacgctcttccgatctctgctcgccacgcatccactggtggtcagatcggaagagcacac

SEQ ID NO: 68 - DNA, 61 bp, synthetic construct
  misc_feature 1..61: note = Synthesized oligonucleotide
  source 1..61: mol_type = other DNA
cgacgctcttccgatctctggccgtgttggtcgtgcttcgggtcagatcggaagagcacac

SEQ ID NO: 69 - DNA, 64 bp, synthetic construct
  misc_feature 1..64: note = Synthesized oligonucleotide
  source 1..64: mol_type = other DNA
cgacgctcttccgatctctgggactccgattgtaggctggcagtgtcagatcggaagagcacac

SEQ ID NO: 70 - DNA, 63 bp, synthetic construct
  misc_feature 1..63: note = Synthesized oligonucleotide
  source 1..63: mol_type = other DNA
cgacgctcttccgatctctgtggaaagtgggtcgctcagtggggtcagatcggaagagcacac

SEQ ID NO: 71 - DNA, 61 bp, synthetic construct
  misc_feature 1..61: note = Synthesized oligonucleotide
  source 1..61: mol_type = other DNA
cgacgctcttccgatctctgtgcgaatgcagccaacacgcagtcagatcggaagagcacac

SEQ ID NO: 72 - DNA, 63 bp, synthetic construct
  misc_feature 1..63: note = Synthesized oligonucleotide
  source 1..63: mol_type = other DNA
cgacgctcttccgatctctgacagctactcgtgcgggaaagcagtcagatcggaagagcacac

SEQ ID NO: 73 - DNA, 64 bp, synthetic construct
  misc_feature 1..64: note = Synthesized oligonucleotide
  source 1..64: mol_type = other DNA
cgacgctcttccgatctctggccggcgaaatcacatgtgtttgcgtcagatcggaagagcacac

SEQ ID NO: 74 - DNA, 61 bp, synthetic construct
  misc_feature 1..61: note = Synthesized oligonucleotide
  source 1..61: mol_type = other DNA
cgacgctcttccgatctctgagtgacgacggtggcttgccagtcagatcggaagagcacac

SEQ ID NO: 75 - DNA, 73 bp, synthetic construct
  misc_feature 1..73: note = Synthesized oligonucleotide
  variation 30..37: note = n is a, c, g, or t
  source 1..73: mol_type = other DNA
aatgatacggcgaccaccgagatctacacnnnnnnnnacactctttccctacacgacgctcttccgatctctg

SEQ ID NO: 76 - DNA, 69 bp, synthetic construct
  misc_feature 1..69: note = Synthesized oligonucleotide
  variation 25..32: note = n is a, c, g, or t
  source 1..69: mol_type = other DNA
caagcagaagacggcatacgagatnnnnnnnngtgactggagttcagacgtgtgctcttccgatctgac

(19) United States
(12) Patent Application Publication
(10) Pub. No.: US 2026/0367074 A1
(43) Pub. Date: Nov. 19, 2026

(54) KIT FOR PROFILING CELL SURFACE GLYCANS ON A SINGLE CELL

(71) Applicant: NATIONAL INSTITUTE OF ADVANCED INDUSTRIAL SCIENCE AND TECHNOLOGY, Tokyo (JP)

(72) Inventor: Hiroaki TATENO, Tsukuba-shi (JP)

(73) Assignee: NATIONAL INSTITUTE OF ADVANCED INDUSTRIAL SCIENCE AND TECHNOLOGY, Tokyo (JP)

(21) Appl. No.: 19/752,974

(22) Filed: Jul. 24, 2026

Related U.S. Application Data
(63) Continuation of application No. 17/913,698, filed on Mar. 15, 2021, now Pat. No. 12,748,105.

(30) Foreign Application Priority Data
Mar. 24, 2020 (JP) 2020-053296

(51) Int. Cl. G01N 33/53; C12Q 1/686; G01N 33/58

(52) U.S. Cl. CPC G01N 33/5308; C12Q 1/686; G01N 33/58; G01N 2400/10

(57) ABSTRACT

A kit for profiling cell surface glycans on a single cell is provided. The kit comprises a mixture of ten or more kinds of glycan-binding substances, each bound to or labeled with a respective nucleic acid. The respective nucleic acid includes an encoding base sequence configured to identify the glycan-binding substance, and at least one adapter sequence configured to amplify the encoding base sequence by PCR. The kit may further comprise a buffer solution configured to serve as a solvent for the glycan-binding substances, maintaining them in a soluble state. This configuration facilitates simultaneous contact with cell surface glycans, physical removal of unbound substances, and isolation of a single intact and living cell having bound substances. The respective nucleic acid may optionally be bound to the glycan-binding substance via a spacer, such as a spacer comprising a cleavable group.

### CROSS-REFERENCE TO RELATED APPLICATIONS

The present application is a continuation application of U.S. patent application Ser. No. 17/913,698 filed Sep. 22, 2022, which is a National Stage Application of PCT/JP2021/010385 filed Mar. 15, 2021, which claims priority of Japanese Patent Application No. 2020-053296 filed Mar. 24, 2020. The disclosures of the prior applications are hereby incorporated by reference herein in their entirety.

### INCORPORATION BY REFERENCE OF SEQUENCE LISTING XML

The Sequence Listing XML filed electronically on Jul. 31, 2026, entitled “Replacement_SQL_26P0157SHG.xml”, created on Jul. 31, 2026, and having a size of 100,269 bytes, is incorporated by reference herein in its entirety. The Sequence Listing XML contains the sequence disclosure for the present application.

### FIELD OF INVENTION

The present invention relates to a method for analyzing a glycan. More specifically, the present invention relates to a method for analyzing a glycan, a kit for analyzing a glycan, and a glycan-binding substance. Priority is claimed on Japanese Patent Application No. 2020-053296, filed Mar. 24, 2020, the content of which is incorporated herein by reference.

### DESCRIPTION OF RELATED ART

Various proteins and lipids are present on the surface of cells. A large number of these have been subjected to glycan modification, and it is known that the surface of cells is covered with a wide variety of glycans.

It is known that glycans have a function such as mediating a cell-cell interaction and change depending on the kind and state of cells. For example, it is known that glycans can be used as cell undifferentiation markers or cancer markers.

In the related art, the analysis of glycans has been carried out by a method of staining cells or the like using a lectin or an antibody, a method of using a liquid chromatography apparatus and a mass spectrometer, a method of using a lectin array in which various kinds of lectins are immobilized on a substrate, and the like (see, for example, Patent Document 1).

### CITATION LIST

### Patent Document

### [Patent Document 1]

PCT International Publication No. WO2010/131641

### SUMMARY OF THE INVENTION

### Technical Problem

However, it may be difficult to grasp the whole picture of glycans by the method of staining cells or the like using a lectin or an antibody. In addition, the method of using a liquid chromatography apparatus and a mass spectrometer requires a great deal of labor and a lot of time and samples.

On the other hand, according to the method of using a lectin array, glycans can be analyzed relatively easily. However, it may be difficult to analyze the glycome of an actual living cell since proteins extracted by destroying a cell are analyzed. In addition, it is necessary to use about 500 ng of proteins for analysis, and thus it is not possible to analyze glycans at a single cell level. For this reason, it is particularly difficult to analyze a tissue section. Further, although a lectin array is produced by using a special spotter, it tends to be difficult to produce a uniform lectin array due to the difference between lots. As a result, it may be difficult to obtain analysis results having good reproducibility by the method using a lectin array. In addition, the analysis of glycans using a lectin array requires a special and expensive scanner for detection. Based on this background, an object of the present invention is to provide a novel technique for analyzing a glycan.

### Solution to Problem

The present invention includes the following aspects.

[1] A method for analyzing a cell surface glycan, the method including:
bringing a glycan-binding substance labeled with a nucleic acid into contact with the cell; and
detecting the nucleic acid labeled to the glycan-binding substance bound to the cell,
in which a kind and a quantity of the nucleic acid correspond to a kind and a quantity of the glycan on the surface of the cell.

[2] The method according to claim 1, further including analyzing a phenotype of the cell or RNA information in the cell, together with the glycan.

[3] The method according to [1] or [2], in which the bringing of the glycan-binding substance labeled with the nucleic acid into contact with the cell is carried out in a buffer solution containing albumin.

[4] The method according to any one of [1] to [3], in which the method is carried out at a single cell level.

[5] The method according to any one of [1] to [4], in which the detection is carried out by real-time quantitative PCR, digital PCR, or sequencing with a next generation sequencer.

[6] A glycan-binding substance labeled with a nucleic acid.

[7] A kit for analyzing a cell surface glycan, the kit including the glycan-binding substance according to [6].

### Advantageous Effects of Invention

According to the present invention, it is possible to provide a novel technique for analyzing a glycan.

### BRIEF DESCRIPTION OF THE DRAWINGS

FIG. 1 is a photographic image showing the results of Coomassie Brilliant Blue (CBB) staining after separating each purified fusion protein by SDS-polyacrylamide gel electrophoresis (SDS-PAGE) in Experimental Example 1.

FIG. 2 is photographic images showing the results of agarose gel electrophoresis in Experimental Example 1.

FIG. 3 is a photographic image showing the results obtained by subjecting specimens from each process of the preparation of nucleic acid-labeled lectin to SDS-PAGE and subjecting them to silver staining, in Experimental Example 2.

FIG. 4 is a photographic image showing the results obtained by subjecting specimens from each process of the preparation of nucleic acid-labeled lectin to SDS-PAGE and subjecting them to silver staining, in Experimental Example 3.

FIG. 5 is a photographic image showing the results obtained by subjecting specimens from each process of the purification of nucleic acid-labeled lectin to SDS-PAGE and subjecting them to silver staining, in Experimental Example 4.

FIG. 6 is a photographic image showing the results obtained by subjecting specimens from each process of the purification of nucleic acid-labeled lectin to SDS-PAGE and subjecting them to silver staining, in Experimental Example 5.

FIG. 7A is a graph showing the results of real-time quantitative PCR in Experimental Example 6.

FIG. 7B is graphs showing the results of flow cytometric analysis in Experimental Example 6.

FIG. 8 is a graph showing the results of real-time quantitative PCR in Experimental Example 7.

FIG. 9A is a graph showing the results of flow cytometric analysis in Experimental Example 8.

FIG. 9B is a graph showing the results of flow cytometric analysis in Experimental Example 8.

FIG. 9C is a graph showing the results of flow cytometric analysis in Experimental Example 8.

FIG. 9D is a graph showing the results of flow cytometric analysis in Experimental Example 8.

FIG. 9E is a graph showing the results of flow cytometric analysis in Experimental Example 8.

FIG. 10 is a graph showing the results of flow cytometric analysis and real-time quantitative PCR in Experimental Example 9.

FIG. 11A is graphs showing the results of flow cytometric analysis in Experimental Example 10.

FIG. 11B is a graph showing the results of real-time quantitative PCR in Experimental Example 10.

FIG. 12 is a graph showing the results of flow cytometric analysis on the lateral axis and the results of real-time quantitative PCR on the vertical axis based on the results of FIG. 11A and FIG. 11B.

FIG. 13 is a graph showing the results of real-time quantitative PCR in Experimental Example 11.

FIG. 14 is a diagram showing the results of Experimental Example 12.

FIG. 15 is a graph showing the results of Experimental Example 13.

FIG. 16 is a graph showing the results of Experimental Example 14.

FIG. 17A is a graph showing the results of Experimental Example 15.

FIG. 17B is a graph showing the results of Experimental Example 15.

FIG. 18A is a graph showing representative results obtained by analyzing a cell surface glycan and RNA in cells at a single cell level in Experimental Example 16.

FIG. 18B is a graph showing representative results obtained by analyzing a cell surface glycan and RNA in cells at a single cell level in Experimental Example 16.

FIG. 19A is a graph showing the results obtained by subjecting the glycan profile of iPS cells induced to differentiate into the nerve to the main component analysis in Experimental Example 17.

FIG. 19B is a graph showing the results obtained by analyzing the expression level of marker genes of iPS cells induced to differentiate into the nerve in Experimental Example 17.

FIG. 20A is a graph showing the results of calculating the correlation coefficient between the amount of the rBC2LCN lectin bound to human iPS cells and the expression level of a group of 27,686 genes and arranging them in descending order of numerical values in Experimental Example 17.

FIG. 20B is a scatter plot showing the amount of the rBC2LCN lectin bound to each cell and the expression level of the POU5F1 gene expression level in each cell in Experimental Example 17.

FIG. 20C is a scatter plot showing the amount of the rBC2LCN lectin bound to each cell and the expression level of the VIM gene expression level in each cell in Experimental Example 17.

FIG. 21A is a graph in which the correlation coefficient between the expression level of the POU5F1 gene in each cell and the binding amount of 39 kinds of lectins is calculated and arranged in descending order in Experimental Example 17.

FIG. 21B is a graph in which the correlation coefficient between the expression level of the OTX2 gene in each cell and the binding amount of 39 kinds of lectins is calculated and arranged in descending order in Experimental Example 17.

### DETAILED DESCRIPTION OF THE INVENTION

[Glycan-Binding Substance Labeled with Nucleic Acid]

In one embodiment, the present invention provides a glycan-binding substance labeled with a nucleic acid. As will be described later, in a case of using the glycan-binding substance of the present embodiment, a cell surface glycan can be easily analyzed with a high sensitivity. The glycan-binding substance is not particularly limited as long as it is a substance that recognizes glycan structure and specifically binds thereto, and examples thereof include a lectin, an antibody, an antibody fragment, and an aptamer. Examples of the antibody fragment include F(ab′)₂, Fab′, Fab, Fv, and scFv.

In the present specification, lectin is defined as a general term referring to proteins having an activity of binding to a glycan. The lectin is not particularly limited, and it is possible to suitably use, for example, the lectins shown in Tables 1 to 5 below. In Tables 1 to 5, “Natural” indicates that the corresponding one is derived from a natural product, and “E. coli” indicates that the corresponding one is a genetically modified product. In addition, “EY Lab.” indicates EY Laboratories Inc., “Wako” indicates FUJIFILM Wako Pure Chemical Corporation, “Seikagaku” indicates SEIKAGAKU CORPORATION, “Vector” indicates Vector Laboratories, Inc., “JOM” indicates J-OIL MILLS, Inc., and “AIST” indicates National Institute of Advanced Industrial Science and Technology. The lectin from the source of supply of “AIST” is prepared by the inventors (Tateno H., et al., Glycome diagnosis of human induced pluripotent stem cells using lectin microarray, J Biol Chem., 286 (23), see 20345-20353, 2011).

In addition, “Sia” indicates sialic acid, “GlcNAc” indicates N-acetyl-glucosamine, “Man” indicates mannose, “Gal” indicates D-galactose, “GalNAc” indicates N-acetyl-galactosamine, “Fuc” indicates L-fucose, “Glc” indicates D-glucose, and “LacNAc” indicates N-acetyl-lactosamine.

The lectin is preferably a recombinant lectin derived from Escherichia coli, which has not been subjected to glycan modification. Further, in order to comprehensively analyze the glycan, it is preferable to use a mix of lectins that recognize Sia, Gal, GlcNAc, Man, Fuc, and GalNAc, which are monosaccharides constituting the glycan.

TABLE 1

| | Name of | Species from which | | Source of | |
|---|---|---|---|---|---|
| Number | lectin | lectin is derived | Origin | supply | Specificity |
| 1 | LFA | Limax flavus | Natural | EY Lab. | Sia |
| 2 | WGA | Triticum | Natural | EY Lab. | (GlcNAc)n, polySia |
| | | vulgaris | | | |
| 3 | PVL | Psathyrella | Natural | Wako | Sia, GlcNAc |
| | | velutina | | | |
| 4 | MAL | Maackia | Natural | Seikagaku | α2-3Sia |
| | | amurensis | | | |
| 5 | MAH | Maackia | Natural | Vector | α2-3Sia |
| | | amurensis | | | |
| 6 | ACG | Agrocybe | Natural | JOM | α2-3Sia |
| | | cylindracea | | | |
| 7 | rACG | Agrocybe | E. coli | AIST | α2-3Sia |
| | | cylindracea | | | |
| 8 | rGal8N | Homo sapiens | E. coli | AIST | α2-3Sia |
| 9 | SNA | Sambucus nigra | Natural | Seikagaku | α2-6Sia |
| 10 | SSA | Sambucus | Natural | Vector | α2-6Sia |
| | | sieboldiana | | | |
| 11 | TJAI | Trichosanthes | Natural | Vector | α2-6Sia |
| | | japonica | | | |
| 12 | rPSL1a | Polyporus | E. coli | AIST | α2-6Sia |
| | | squamosus | | | |
| 13 | PHAL | Phaseolus | Natural | Seikagaku | GlcNAcβ1-6Man |
| | | vulgaris | | | (Tetraantenna) |
| 14 | DSA | Datura | Natural | Seikagaku | GlcNAcβ1-6Man |
| | | stramonium | | | (Tetraantenna) |
| 15 | TxLcI | Tulipa | Natural | JOM | Galactosylated N-glycans |
| | | gesneriana | | | up to triantenna |
| 16 | ECA | Erythrina | Natural | Seikagaku | βGal |
| | | cristagalli | | | |
| 17 | RCA120 | Ricinus | Natural | Vector | βGal |
| | | communis | | | |
| 18 | rGal7 | Homo sapiens | E. coli | AIST | Type1 LacNAc, chondroitin |
| | | | | | polymer |
| 19 | rGal9N | Homo sapiens | E. coli | AIST | GalNAcα1-4Gal (A), |
| | | | | | PolyLacNAc |
| 20 | rGal9C | Homo sapiens | E. coli | AIST | PolyLacNAc, Branched |
| | | | | | LacNAc |

TABLE 2

| | Name of | Species from which | | Source of | |
|---|---|---|---|---|---|
| Number | lectin | lectin is derived | Origin | supply | Specificity |
| 21 | rC14 | Gallus gallus | E. coli | AIST | Branched LacNAc |
| | | domesticus | | | |
| 22 | rDiscoidin II | Dictyostelium | E. coli | AIST | LacNAc, Galβ1-3GalNAc |
| | | dicodeum | | | (T), GalNAc (Tn) |
| 23 | BPL | Bauhinia | Natural | Vector | Galβ1-3GlcNAc(GalNAc), |
| | | purpurea alba | | | α/βGalNAc |
| 24 | rCGL2 | Homo sapiens | E. coli | AIST | GalNAcα1-3Gal (A), |
| | | | | | PolyLacNAc |
| 25 | PHAE | Phaseolus | Natural | Vector | bisecting GlcNAc |
| | | vulgaris | | | |
| 26 | GSLII | Griffonia | Natural | Vector | GlcNAcβ1-4Man |
| | | simplicifolia | | | |
| 27 | rSRL | Sclerotium | E. coli | AIST | Core1,3, agalacto N-glycan |
| | | rolfsii | | | |
| 28 | UDA | Urtica dioica | Natural | Vector | (GlcNAc)n |
| 29 | PWM | Phytolacca | Natural | Vector | (GlcNAc)n |
| | | americana | | | |
| 30 | rF17AG | Escherichia coli | E. coli | AIST | GlcNAc |
| 31 | rGRFT | Griffithia sp. | E. coli | AIST | Man |
| 32 | NPA | Narcissus | Natural | Seikagaku | Mana1-3Man |
| | | pseudonarcissus | | | |
| 33 | ConA | Canavalia | Natural | Vector | M3, Manα1-2Manα1- |
| | | ensiformis | | | 3(Manα1-6)Man, GlcNAcβ1- |
| | | | | | 2Manα1-3(Manα1-6)Man |
| 34 | GNA | Galanthus | Natural | Vector | Manα1-3Man, Manα1- |
| | | nivalis | | | 6Man |
| 35 | HHL | Hippeastrum | Natural | Vector | Manα1-3Man, Manα1- |
| | | hybrid | | | 6Man |
| 36 | ASA | Allium sativum | Natural | JOM | Galβ1-4GlcNAcβ1-2Man |
| 37 | DBAI | Dioscorea | Natural | JOM | High-man |
| | | batatas | | | |
| 38 | CCA | Castanea | Natural | JOM | Galactosylated N-glycans |
| | | crenata | | | up to triantenna |
| 39 | Heltuba | Helianthus | Natural | JOM | Manα1-3Man |
| | | tuberosus | | | |
| 40 | rHeltuba | Helianthus | E. coli | AIST | Manα1-3Man |
| | | tuberosus | | | |

TABLE 3

| | Name of | Species from which | | Source of | |
|---|---|---|---|---|---|
| Number | lectin | lectin is derived | Origin | supply | Specificity |
| 41 | ADA | Allomyrina | Natural | JOM | α2-6Sia, Forssman, A, B |
| | | dichtoma | | | |
| 42 | VVAII | Vicia villosa | Natural | JOM | Man, Agalacto |
| 43 | rOrysata | Oryza sativa | E. coli | AIST | Manα1-3Man, Highman, |
| | | | | | biantenna |
| 44 | rPALa | Phlebodium | E. coli | AIST | Man5, biantenna |
| | | aureum | | | |
| 45 | rBanana | Musa | E. coli | AIST | Manα1-2Manα1-3(6)Man |
| | | acuminata | | | |
| 46 | rCalsepa | Calystegia | E. coli | AIST | Biantenna with bisecting |
| | | sepium | | | GlcNAc |
| 47 | rRSL | Ralstonia | E. coli | AIST | αMan, α1-2Fuc (H), α1- |
| | | solanacearum | | | 3Fuc (Lex), α1-4Fuc (Lea) |
| 48 | rBC2LA | Burkholderia | E. coli | AIST | αMan, High-man |
| | | cenocepacia | | | |
| 49 | AOL | Aspergillus | Natural | Vector | α1-2Fuc (H), α1-3Fuc |
| | | oryzae | | | (Lex), α1-3Fuc(Lea) |
| 50 | AAL | Aleuria | Natural | Vector | α1-2Fuc (H), α1-3Fuc |
| | | aurantia | | | (Lex), α1-4Fuc (Lea) |
| 51 | rAAL | Aleuria | E. coli | AIST | α1-2Fuc (H), α1-3Fuc |
| | | aurantia | | | (Lex), α1-3Fuc (Lea) |
| 52 | rPAIIL | Pseudomonas | E. coli | AIST | αMan, α1-2Fuc (H), α1- |
| | | aeruginosa | | | 3Fuc (Lex), α1-4Fuc (Lea) |
| 53 | rRSIIL | Ralstonia | E. coli | AIST | α1-2Fuc (H), α1-3Fuc |
| | | solanacearum | | | (Lex), α1-3Fuc (Lea) |
| 54 | rPTL | Pholiota | E. coli | AIST | α1-6Fuc |
| | | terrestris | | | |
| 55 | PSA | Pisum sativum | Natural | Seikagaku | α1-6Fuc up to biantenna |
| 56 | LCA | Lens culinaris | Natural | Vector | α1-6Fuc up to biantenna |
| 57 | rAOL | Aspergillus | E. coli | AIST | α1-2Fuc (H), α1-3Fuc |
| | | oryzae | | | (Lex), α1-3Fuc (Lea) |
| 58 | rBC2LCN | Burkholderia | E. coli | AIST | Fuc α1-2Galβ1-3GlcNAc |
| | | cenocepacia | | | (GalNAc) |
| 59 | LTL | Lotus | Natural | Seikagaku | Lex, Ley |
| | | tetragonolobus | | | |
| 60 | UEAI | Ulex europaeus | Natural | Vector | α1-2Fuc |

TABLE 4

| | Name of | Species from which | | Source of | |
|---|---|---|---|---|---|
| Number | lectin | lectin is derived | Origin | supply | Specificity |
| 61 | TJAII | Trichosanthes | Natural | Vector | α1-2Fuc |
| | | japonica | | | |
| 62 | MCA | Momordica | Natural | JOM | α1-2Fuc |
| | | charantia | | | |
| 63 | GSLI | Griffonia | Natural | Seikagaku | αGalNAc (A, Tn), αGal (B) |
| | | simplicifolia | | | |
| 64 | PTLI | Psophocarpus | Natural | Tokyo | αGalNAc (A, Tn) |
| | | tetragonolobus | | Kasei | |
| 65 | GSLIA4 | Griffonia | Natural | EY Lab. | αGalNAc (A, Tn) |
| | | simplicifolia | | | |
| 66 | rGC2 | Geodia | E. coli | AIST | α1-2Fuc (H), αGalNAc (A), |
| | | cydonium | | | αGal (B) |
| 67 | GSLIB4 | Griffonia | Natural | Vector | αGal (B) |
| | | simplicifolia | | | |
| 68 | rMOA | Marasmius | E. coli | AIST | αGal (B) |
| | | oreades | | | |
| 69 | EEL | Euonymus | Natural | Vector | αGal (B) |
| | | europaeus | | | |
| 70 | rPAIL | Pseudomonas | E. coli | AIST | α,βGal, αGalNAc (Tn) |
| | | aeruginosa | | | |
| 71 | LEL | Lycopersicon | Natural | Vector | Polylactosamine, (GlcNAc)n |
| | | esculentum | | | |
| 72 | STL | Solanum | Natural | Seikagaku | Polylactosamine, (GlcNAc)n |
| | | tuberosum | | | |
| 73 | rGal3C | Homo sapiens | E. coli | AIST | LacNAc, polylactosamine |
| 74 | rLSLN | Laetiporus | E. coli | AIST | LacNAc, polylactosamine |
| | | sulphureus | | | |
| 75 | rCGL3 | Coprinopsis | E. coli | AIST | LacDiNAc |
| | | cinerea | | | |
| 76 | PNA | Arachis | Natural | Vector | Galβ1-3GalNAc (T) |
| | | hypogaea | | | |
| 77 | ACA | Amaranthus | Natural | Vector | Galβ1-3GalNAc (T) |
| | | caudatus | | | |
| 78 | HEA | Hericium | Natural | JOM | Galβ1-3GalNAc (T) |
| | | erinaceum | | | |
| 79 | ABA | Agarics | Natural | Vector | Galβ1-3GalNAc (T), GlcNAc |
| | | bisporus | | | |
| 80 | Jacalin | Artocarpus | Natural | Seikagaku | Galβ1-3GalNAc (T), |
| | | integrifolia | | | GalNAcα (Tn) |

TABLE 5

| | Name of | Species from which | | Source of | |
|---|---|---|---|---|---|
| Number | lectin | lectin is derived | Origin | supply | Specificity |
| 81 | MPA | Maclura | Natural | Seikagaku | Galβ1-3GalNAc (T), |
| | | pomifera | | | GalNAcα (Tn) |
| 82 | HPA | Helix pomatia | Natural | Seikagaku | αGalNAc (A, Tn) |
| 83 | VVA | Vicia villosa | Natural | Vector | α,βGalNAc (A, Tn, |
| | | | | | LacDiNAc) |
| 84 | DBA | Dolichos biflorus | Natural | Vector | α,βGalNAc (A, Tn, |
| | | | | | LacDiNAc) |
| 85 | SBA | Glycine max | Natural | EY Lab. | α,βGalNAc (A, Tn, |
| | | | | | LacDiNAc) |
| 86 | rPPL | Pleurocybella | E. coli | AIST | α,βGalNAc (A, Tn, |
| | | porrigens | | | LacDiNAc) |
| 87 | rCNL | Clitocybe | E. coli | AIST | α,βGalNAc (A, Tn, |
| | | nebularis | | | LacDiNAc) |
| 88 | rXCL | Xerocomus | E. coli | AIST | Corel,3, agalacto N-glycan |
| | | chrysenteron | | | |
| 89 | VVA I | Vicia villosa | Natural | JOM | GalNAcβ1-3(4)Gal |
| 90 | WFA | Wisteria | Natural | Vector | Terminal GalNAc, |
| | | floribunda | | | LacDiNAc |
| 91 | rABA | Agarics | E. coli | AIST | Galβ1-3GalNAc (T), |
| | | bisporus | | | GlcNAc |
| 92 | rDiscoidin I | Dictyostelium | E. coli | AIST | Gal |
| | | Discodeum | | | |
| 93 | DBAIII | Dioscorea | Natural | JOM | Maltose |
| | | batatas | | | |
| 94 | rMalectin | Homo sapiens | E. coli | AIST | Glcα1-2Glc |
| 95 | CSA | Oncorhynchus | Natural | JOM | Rhamnose, Galα1-4Gal |
| | | keta | | | |
| 96 | FLAG- | Lumbricus | E. coli | AIST | 6-sulfo-Gal |
| | EW29Ch-E20K | terrestris | | | |

The nucleic acid with which the glycan-binding substance is labeled may be, for example, a cyclic nucleic acid. It may be, for example, a single-stranded nucleic acid fragment or may be, for example, a double-stranded nucleic acid fragment. Examples of the circular nucleic acid include plasmid DNA. In addition, the nucleic acid with which the glycan-binding substance is labeled may be DNA or RNA; however, it is preferably DNA from the viewpoint of stability.

It is preferable to label a specific kind of glycan-binding substance with a specific kind of nucleic acid. For example, in a case of causing the kind of glycan-binding substance to correspond to the base sequence of the nucleic acid with which the glycan-binding substance is labeled, it is possible to encode the glycan-binding substance. As a result, the base sequence of the nucleic acid with which the glycan-binding substance is labeled is preferably a base sequence that is not present in nature. In a case of detecting the base sequence specific to the nucleic acid with which the glycan-binding substance is labeled, it is possible to detect the presence of the glycan-binding substance corresponding thereto.

Further, in a case of amplifying the nucleic acid bonded to the glycan-binding substance by PCR or the like, it is possible to amplify the signal that indicates the presence of the glycan-binding substance. As a result, it is possible to increase, for example, the detection sensitivity.

In a case where the nucleic acid with which the glycan-binding substance is labeled is a single-stranded nucleic acid fragment or a double-stranded nucleic acid fragment, the length thereof does not affect the binding of the glycan and is not particularly limited as long as it can retain the information that indicates the corresponding glycan-binding substance. For example, it may be several tens of bases (or base pairs) to several tens of kilobases (or base pairs). Further, the nucleic acid may be circular DNA such as a plasmid.

As will be described later, the nucleic acid with which the glycan-binding substance is labeled may be detected by real-time quantitative PCR, digital PCR, or sequencing by a next generation sequencer.

Therefore, it is preferable that the nucleic acid with which the glycan-binding substance is labeled further have a base sequence region to which a PCR primer can hybridize. In addition, in a case where the nucleic acid with which the glycan-binding substance is labeled is detected by sequencing with a next generation sequencer, the nucleic acid with which the glycan-binding substance is labeled preferably further has a base sequence that enables the pretreatment for the next generation sequencing such as bridge PCR or emulsion PCR.

The length of the nucleic acid with which the glycan-binding substance is labeled is particularly preferably 50 to 100 bases. Among these, the base sequence for encoding the glycan-binding substance is 10 to 30 bases, and it is preferable to add an adapter sequence of 10 to 30 bases to each of the 5′ side and the 3′ side thereof. The base sequence for encoding the glycan-binding substance is selected so that the base is not biased.

In addition to the sequence complementary to the adapter sequence, the base sequence of the PCR primer preferably includes a base sequence (5 to 10 bases) for identifying various kinds of cells and a base sequence (20 to 30 bases) for hybridizing to a flow cell in the next generation sequencing.

As will be described later in Examples, the binding of a nucleic acid to the glycan-binding substance may be carried out, for example, by linking a nucleic acid-binding domain to the glycan-binding substance and binding the nucleic acid to the nucleic acid binding domain, may be carried out by using a chemical linker to bind the glycan-binding substance to a nucleic acid, or may be carried out by using the click reaction to bind the glycan-binding substance to a nucleic acid.

In order for a chemical linker to be capable of being usable, a functional group such as an amino group or an SH group may be introduced into the nucleic acid. Further, in order for the click reaction to be capable of being usable, an azide group, an alkyne group, or the like may be introduced into the nucleic acid. The introduction of this group can be carried out by chemical synthesis of nucleic acid, or the like.

Further, a spacer may be present between the glycan-binding substance and the nucleic acid. The spacer is not particularly limited, and examples thereof include a polyethylene glycol chain, a polyacrylamide chain, a polyester chain, a polyurethane chain, and a copolymer thereof. The spacer may be derived from a chemical linker.

The spacer may also contain a cleavable group. For example, as will be described later in Examples, in a case where the spacer contains a group that is cleavable upon irradiation with light, it is possible to detach the nucleic acid from the glycan-binding substance and recover it by irradiating the glycan-binding substance labeled with a nucleic acid with light.

### [Method of Analyzing a Glycan]

In one embodiment, the present invention provides a method for analyzing a cell surface glycan, the method including bringing glycan-binding substance labeled with a nucleic acid into contact with the cell and detecting the nucleic acid labeled to the glycan-binding substance bound to the cell, in which a kind and a quantity of the nucleic acid correspond to a kind and a quantity of the glycan on the surface of the cell.

The cell is not particularly limited, and examples thereof include a microorganism (a virus, a bacterium, a fungus), an insect cell, a plant cell, and an animal cell. Further, a tissue section or the like can also be used as a specimen. Regarding the cell or the tissue section, the cell may be in a state of being alive or fixed.

In the method of the present embodiment, first, a glycan-binding substance labeled with a nucleic acid is brought into contact with cells to be analyzed. As the glycan-binding substance labeled with a nucleic acid, the above-described substance can be used.

One kind of glycan-binding substance labeled with a nucleic acid may be singly brought into contact with cells, or two or more kinds thereof may be mixed and brought into contact with cells. In a case of simultaneously bringing various kinds of glycan-binding substances labeled with a nucleic acid into contact with cells, it is possible to comprehensively analyze the glycan structures on the surface of cells.

The bringing of the glycan-binding substance labeled with a nucleic acid into contact with cells can be carried out by mixing the cell and the glycan-binding substance labeled with a nucleic acid, in a solution such as in a culture medium, physiological saline, or a buffer solution.

Among the above, the bringing of the glycan-binding substance labeled with a nucleic acid into contact with cells is preferably carried out in a buffer solution containing albumin. As will be described later in Examples, the detection signal can be significantly enhanced by carrying out the bringing of the glycan-binding substance labeled with a nucleic acid into contact with cells in a buffer solution containing albumin.

Examples of the buffer solution include a Tris buffer solution and phosphate-buffered saline. Examples of the composition of the phosphate-buffered saline include NaCl 137 mmol/L, KCl 2.7 mmol/L, Na₂HPO₄ 10 mmol/L, and KH₂PO₄ 1.76 mmol/L. Further, it is preferable that the pH of the phosphate-buffered saline be adjusted to about 7.4.

As the albumin, bovine serum albumin, human serum albumin, and the like can be used. The albumin may be a recombinant thereof. The concentration of albumin in the buffer solution is preferably about 0.1 to 10% by mass.

Regarding the amount of the glycan-binding substance labeled with a nucleic acid to be brought into contact with the cells to be analyzed, it suffices that one or more molecules with respect to one kind of glycan-binding substance labeled with a nucleic acid be brought into contact per a single cell, and it is preferable that the amount be such that the cell surface glycan can be saturated.

In addition, the time required for the glycan-binding substance labeled with a nucleic acid to be brought into contact with the cell to be analyzed is not particularly limited as long as it is a time sufficient for the glycan-binding substance to bind to the glycan on the surface of the cells to be analyzed. It may be, for example, about 10 minutes to 24 hours, for example, may be about 10 minutes to 8 hours, for example, may be about 10 minutes to 3 hours, and for example, may be about 1 hour. In addition, the temperature at which the glycan-binding substance labeled with a nucleic acid is brought into contact is not particularly limited as long as the glycan-binding substance binds to the cell surface glycan to be analyzed, and it may be, for example, about 4 °C to 37 °C.

As a result of bringing the glycan-binding substance labeled with a nucleic acid into contact with the cells to be analyzed, the glycan-binding substance binds to the cell surface glycan to be analyzed. Here, it is preferable to remove the unreacted glycan-binding substance. The unreacted glycan-binding substance can be removed, for example, by repeating the operation of adding a buffer solution and centrifuging cells to remove the supernatant one to several times.

Subsequently, the nucleic acid labeled to the glycan-binding substance bound to the cell is detected. Here, the detection of the nucleic acid may be carried out in a state where the glycan-binding substance labeled with a nucleic acid is bound to the cell, may be carried out after the glycan-binding substance labeled with a nucleic acid is dissociated from the cell and further the glycan-binding substance labeled with a nucleic acid is separated from the cell, or may be carried out after a nucleic acid is detached and recovered from the glycan-binding substance labeled with the nucleic acid.

Examples of the method of dissociating the glycan-binding substance labeled with a nucleic acid from the cell include a method of reacting a cell with sugar that competes with the glycan-binding substance, a method of allowing a surfactant to act to dissociate the binding between the glycan-binding substance and the cell surface glycan, a method of changing the pH to dissociate the binding between the glycan-binding substance and the cell surface glycan, and a method of allowing a reducing agent to act to dissociate the binding between the glycan-binding substance and the glycan on the surface of the cell.

Further, as described above, in a case where a cleavable group is introduced between the nucleic acid and the glycan-binding substance, the nucleic acid can be detached and recovered from the glycan-binding substance labeled with a nucleic acid. For example, in a case where a group that can be cleavable upon irradiation with light is introduced between the nucleic acid and the glycan-binding substance in advance, the nucleic acid can be detached from the glycan-binding substance labeled with a nucleic acid by irradiating with light. Then, it is possible to recover the nucleic acid, for example, by carrying out centrifugal separation to recover the supernatant.

The detection of the nucleic acid labeled to the glycan-binding substance bound to the cell may be carried out by, for example, real-time quantitative PCR, digital PCR, or sequencing with a next generation sequencer. By amplifying the nucleic acid by PCR or the like, the detection signal can be amplified, and the detection sensitivity can be increased. Here, a kind and a quantity of the nucleic acid detected corresponds to a kind and a quantity of the cell surface glycan.

Here, the kind of the nucleic acid is the kind of the base sequence of the nucleic acid labeled to the glycan-binding substance. In a case of specifying the kind of the nucleic acid, it is possible to specify the kind of the glycan-binding substance to which the nucleic acid is labeled. As a result, in a case of specifying the base sequence of the nucleic acid, it is possible to specify the structure of the cell surface glycan to be analyzed. In addition, the amount of the nucleic acid having a specific base sequence corresponds to the amount of the glycan-binding substance bound to the cells to be analyzed. That is, the amount of the nucleic acid having a specific base sequence corresponds to the amount of the specific glycan structure present on the surface of the cells to be analyzed. This makes it possible to quantitatively analyze the kind of structure of the glycan and the quantity of glycan present on the surface of the cells to be analyzed.

Real-time quantitative PCR, digital PCR, and next generation sequencing can be carried out using general-purpose devices. Accordingly, in a case where these devices are already present, it is not necessary to prepare a new special device for the analysis of glycans.

Further, as will be described later in Examples, since the method of the present embodiment has high detection sensitivity, it is also possible to analyze a cell surface glycan at a single cell level. Here, to analyze a cell surface glycan at a single cell level means that a single cell is used as a specimen, a glycan-binding substance labeled with a nucleic acid is brought into contact with the specimen, the nucleic acid labeled to the glycan-binding substance bound is detected, and the kind and quantity of the cell surface glycan are specified. The analysis of glycan at a single cell level could not be carried out by the methods in the related art.

Further, in the method of the present embodiment, cells are still alive even after the nucleic acid is recovered from the surface of the cells. Therefore, it is possible to analyze the cell surface glycan and simultaneously analyze the phenotype of the cell or the RNA information in the cell.

Examples of the phenotype of the cell include cell morphology, proliferation ability, differentiation ability, infiltration ability, tumor-forming ability, and the expression of marker protein. In addition, it is also possible to analyze the intracellular genetic information or the phenotype of cells at a single cell level. Examples of the RNA information in the cell include base sequence information or expression level information on mRNA, microRNA, 16S rRNA, non-coding RNA, and the like.

### [Kit]

In one embodiment, the present invention provides a kit for analyzing a cell surface glycan, which includes the above-described glycan-binding substance labeled with a nucleic acid. In a case of using the kit of the present embodiment, it is possible to suitably carry out the analysis of a cell surface glycan.

The kit of the present embodiment may contain one kind of glycan-binding substance labeled with a nucleic acid, or it may contain two or more kinds thereof, for example, 10 kinds or more thereof, for example, 30 kinds or more thereof, for example, 50 kinds or more thereof, or for example, 100 kinds or more thereof. In a case where the kit of the present embodiment contains various kinds of glycan-binding substances labeled with a nucleic acid, it is easy to comprehensively analyze the structures of the cell surface glycan, which is preferable.

The kit of the present embodiment may further contain a buffer solution containing albumin as a solvent in which the glycan-binding substance labeled with a nucleic acid is brought into contact with the cells to be analyzed. The buffer solution containing albumin is the same as that described above. As will be described later in Examples, it is possible to suppress the background in a case in which bringing the glycan-binding substance labeled with a nucleic acid into contact with the cells is carried out in a buffer solution containing albumin.

In addition, the kit of the present embodiment may further contain a primer for detecting a nucleic acid bonded to the glycan-binding substance. The nucleic acid bonded to the glycan-binding substance can be detected by real-time quantitative PCR, digital PCR, next generation sequencing, or the like using the above primer.

### EXAMPLES

Next, the present invention will be described in more detail by showing Examples; however, the present invention is not limited to Examples below.

### [Lectin]

The lectins shown in Table 6 below were used. In addition, in a case where each lectin was labeled with a nucleic acid, it was labeled with a nucleic acid consisting of the base sequence set forth in the SEQ ID NO shown in Table 6 below. In Table 6, “Seikagaku” indicates SEIKAGAKU CORPORATION, “Vector” indicates Vector Laboratories, Inc., “AIST” indicates National Institute of Advanced Industrial Science and Technology, “Wako” indicates FUJIFILM Wako Pure Chemical Corporation, and “JOM” indicates J-OIL MILLS, Inc. Further, “For real-time PCR” indicates that it is SEQ ID NO of the base sequence used for the real-time PCR analysis in the experimental examples described later, and “For next generation sequencing” indicates that it is SEQ ID NO of the base sequence used for the next generation sequencing in the experimental examples described later.

TABLE 6

| | | | SEQ ID NO of base sequence | |
|---|---|---|---|---|
| | SEQ ID NO of | | of labeled nucleic acid | |
| | amino acid | | | For next |
| Name of | sequence | Source of | For real- | generation |
| lectin | of lectin | supply | time PCR | sequencing |
| SNA | — | Seikagaku | 16 | 17 |
| ConA | — | Vector | 23 | 24 |
| GSLII | — | Vector | 18 | 19 |
| rBC2LCN | 1 | AIST | 8 | 9 |
| rAAL | 20 | AIST | 21 | 22 |
| rABA | 10 | AIST | 11 | 12 |
| rLSLN | 13 | AIST | 14 | 15 |
| rPSL1a | — | AIST | 25 | 26 |
| rDiscoidinI | — | AIST | 27 | 28 |
| rF17AG | — | AIST | 29 | 30 |
| rPVL | — | Wako | 31 | 32 |
| rCGL2 | — | AIST | 33 | 34 |
| rPAIL | — | AIST | 35 | 36 |
| rPPL | — | AIST | 37 | 38 |
| rRSIIL | — | AIST | 39 | 40 |
| rCNL | — | AIST | 41 | 42 |
| WFA | — | Vector | 43 | 44 |
| HPA | — | Seikagaku | 45 | 46 |
| SSA | — | Vector | 47 | 48 |
| rOrysata | — | AIST | 49 | 50 |
| rPALa | — | AIST | 51 | 52 |
| rDiscoidinII | — | AIST | 53 | 54 |
| CSA | — | JOM | 55 | 56 |
| rGRFT | — | AIST | 57 | 58 |
| rSRL | — | AIST | 59 | 60 |
| rAOL | — | AIST | — | 61 |
| rBanana | — | AIST | — | 62 |
| rBC2LA | — | AIST | — | 63 |
| rC14 | — | AIST | — | 64 |
| rCalsepa | — | AIST | — | 65 |
| rGal3C | — | AIST | — | 66 |
| rGC2 | — | AIST | — | 67 |
| rMalectin | — | AIST | — | 68 |
| rMOA | — | AIST | — | 69 |
| rPTL | — | AIST | — | 70 |
| rRSL | — | AIST | — | 71 |
| TJAI | — | Vector | — | 72 |
| TJAII | — | Vector | — | 73 |
| UEAI | — | Vector | — | 74 |

### Experimental Example 1

### (Preparation of Nucleic Acid-Labeled Lectin 1)

A nucleic acid-labeled lectin was prepared. First, a fusion protein of a lectin and a nucleic acid binding domain was prepared. A BC2LCN lectin was used as the lectin. The amino acid sequence of BC2LCN lectin is set forth in SEQ ID NO: 1.

Further, as the nucleic acid binding domain, the following peptides were used; a peptide having four consecutive arginine residues (SEQ ID NO: 2, hereinafter referred to as “R4”), a peptide having five consecutive arginine residues (SEQ ID NO: 3, hereinafter referred to as “R5”), a peptide having six consecutive arginine residues (SEQ ID NO: 4, hereinafter referred to as “R6”), a peptide having seven consecutive arginine residues (SEQ ID NO: 5, hereinafter referred to as “R7”), a peptide having ten consecutive arginine residues (SEQ ID NO: 6, hereinafter referred to as “R10”), and an HMG Box A domain of mitochondrial transcription factor A (TFAM) (SEQ ID NO: 7, hereinafter referred to as “TFAM”). It is to be noted that it is known that arginine is basic and easily binds to a nucleic acid having a phosphoric acid moiety.

First, a fusion protein of a recombinant BC2LCN lectin (hereinafter referred to as “rBC2LCN”) in which each nucleic acid binding domain was bound to the C-terminal side was expressed and purified in Escherichia coli. A FLAG tag was introduced on the N-terminal side of each fusion protein.

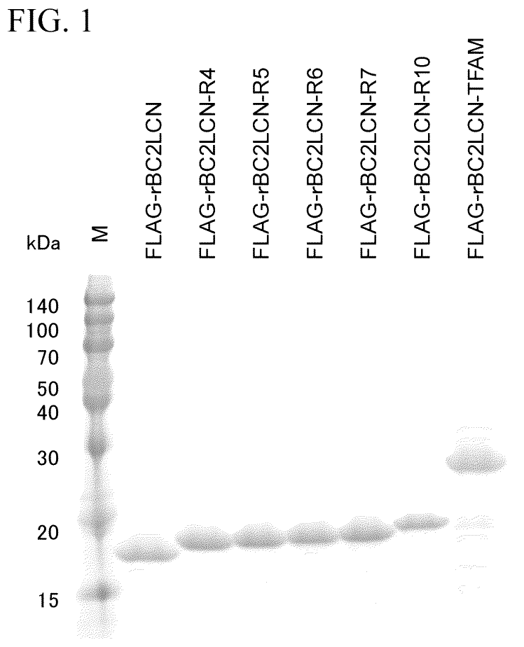

FIG. 1 is a photographic image showing the results of Coomassie Brilliant Blue (CBB) staining after separating each purified fusion protein by SDS-polyacrylamide gel electrophoresis (SDS-PAGE).

In FIG. 1, “M” indicates a molecular weight marker, “FLAG-rBC2LCN” indicates a fusion protein having no nucleic acid binding domain, “FLAG-rBC2LCN-R4” indicates a fusion protein linked with “R4”. “FLAG-rBC2LCN-R5” indicates a fusion protein linked with “R5”, “FLAG-rBC2LCN-R6” indicates a fusion protein linked with “R6”, “FLAG-rBC2LCN-R7” indicates a fusion protein linked with “R7”, “FLAG-rBC2LCN-R10” indicates a fusion protein linked with “R10”, and “FLAG-rBC2LCN-TFAM” indicates a fusion protein linked with “TEAM”.

Subsequently, the binding property of each purified fusion protein to the plasmid DNA (pCR2.1) was analyzed. Specifically, 1 μg of the plasmid DNA (pCR2.1) was mixed with 1, 2, 3, 4, or 5 μg of each fusion protein and subjected to agarose gel electrophoresis.

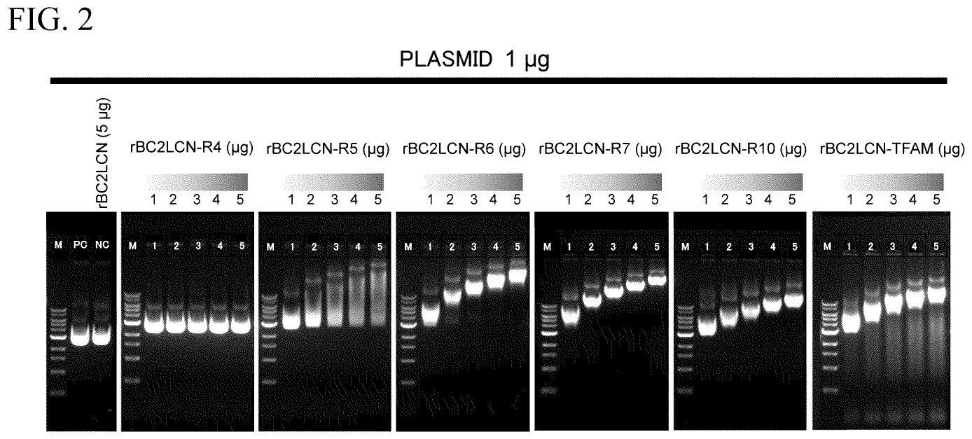

FIG. 2 is photographic images showing the results of agarose gel electrophoresis. In FIG. 2, “M” indicates a molecular weight marker, “rBC2LCN” indicates a fusion protein having no nucleic acid binding domain, “rBC2LCN-R4” indicates a fusion protein linked with “R4”. “rBC2LCN-R5” indicates a fusion protein linked with “R5”, “rBC2LCN-R6” indicates a fusion protein linked with “R6”, “rBC2LCN-R7” indicates a fusion protein linked with “R7”, “rBC2LCN-R10” indicates a fusion protein linked with “R10”, and “rBC2LCN-TFAM” indicates a fusion protein linked with “TFAM”.

As a result, it was revealed that the fusion protein fused with 5 or more arginine residues or TFAM binds to the plasmid DNA. Therefore, it was revealed that a nucleic acid-labeled lectin can be prepared by mixing a nucleic acid with a fusion protein of a lectin and a nucleic acid binding domain.

### Experimental Example 2

### (Preparation of Nucleic Acid-Labeled Lectin 2)

A nucleic acid-labeled lectin was prepared using a commercially available kit (a Protein-oligo conjugation kit, catalog number: “S-9011-1”, Solulink, Inc.).

In this kit, first, N-succinimidyl-4-formylbenzamide (hereinafter, referred to as “S-4FB”) is bonded to an amino group of an oligonucleotide, in which the 5′ terminal or 3′ terminal is modified with the amino group, to prepare a 4-formylbenzamide (4FB)-modified oligonucleotide (a “4FB-oligonucleotide”).

Further, succinimidyl-4-hydrazinonicotinate acetone hydrazone (hereinafter, referred to as “S-HyNic”) is bonded to a protein to prepare a hydrazinonicotinate acetone hydrazone (HyNic)-modified protein (a “HyNic-protein”).

Subsequently, in a case where the above 4FB-oligonucleotide and the above HyNic-protein are mixed, both can be covalently bonded to obtain a nucleic acid-labeled protein.

In the present experimental example, an oligonucleotide was bonded to rBC2LCN according to the instruction manual of the kit to prepare a nucleic acid-labeled lectin. rBC2LCN was used as the lectin. Further, an oligonucleotide having a base sequence set forth in SEQ ID NO: 8 was used as the nucleic acid.

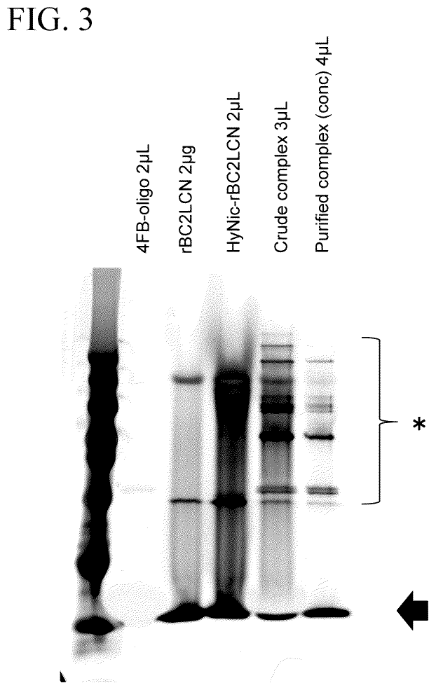

FIG. 3 is a photographic image showing the results obtained by subjecting specimens from each process of the preparation of nucleic acid-labeled lectin to SDS-PAGE and subjecting them to silver staining. In FIG. 3, “4FB-oligo” indicates a 4FB-modified oligonucleotide, “HyNic-rBC2LCN” indicates a HyNic-modified rBC2LCN, “Crude complex” indicates an unpurified reactant, and “Purified complex (conc)” indicates a purified and concentrated reactant. Further, “*” indicates rBC2LCN to which the oligonucleotide is bonded, and the arrow indicates rBC2LCN to which the oligonucleotide is not bonded.

As a result, rBC2LCN having an increased molecular weight due to the bonding of oligonucleotides was detected in the final product (the Purified complex (conc)). From this result, it was revealed that the nucleic acid-labeled lectin can be prepared by using a commercially available kit.

### Experimental Example 3

### (Preparation of Nucleic Acid-Labeled Lectin 3)

A nucleic acid-labeled lectin was prepared using the click reaction between an azide group and an alkyne group. Specifically, first, rBC2LCN was reacted with each of 15-fold, 30-fold, and 50-fold molar concentrations of dibenzocyclooctyne-N-hydroxysuccinimidyl ester (NHS-DBCO) at room temperature for 1 hour to be subjected to DBCO modification. Subsequently, each rBC2LCN subjected to DBCO modification was mixed with a 10-fold molar concentration of an azinated oligonucleotide (SEQ ID NO: 8) and reacted at 4 °C overnight. The oligonucleotide was subjected to the azination modification at the 5′ terminal.

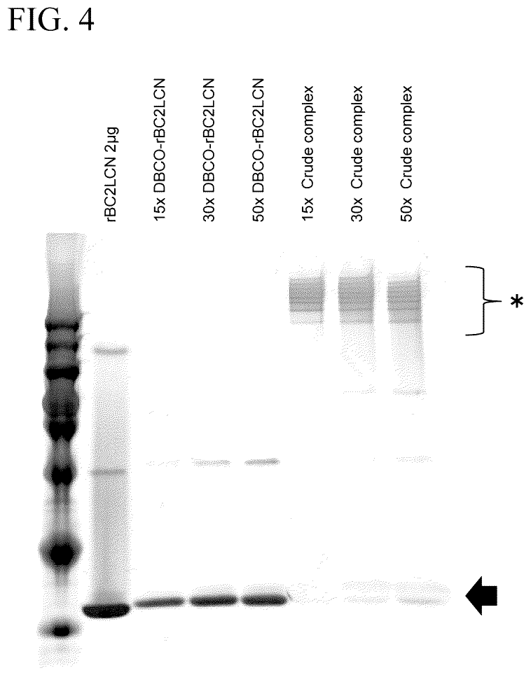

FIG. 4 is a photographic image showing the results obtained by subjecting specimens from each process to SDS-PAGE and subjecting them to silver staining. In FIG. 4, “DBCO-rBC2LCN” indicates rBC2LCN subjected to DBCO modification, “15×”, “30×”, and “50×” indicate that the corresponding result is obtained by the reaction with each of 15-fold, 30-fold, and 50-fold molar concentrations of NHS-DBCO, and “Crude complex” indicates an unpurified reactant. Further, “*” indicates rBC2LCN to which the oligonucleotide is bonded, and the arrow indicates rBC2LCN to which the oligonucleotide is not bonded.

As a result, rBC2LCN having an increased molecular weight due to the bonding of oligonucleotides was detected. From this result, it was revealed that the nucleic acid-labeled lectin can be efficiently prepared by the click reaction.

### Experimental Example 4

### (Preparation of Nucleic Acid-Labeled Lectin 4)

The click reaction between an azide group and an alkyne group was used to prepare a nucleic acid-labeled lectin. Specifically, first, rBC2LCN was reacted with a 2-fold molar concentration of dibenzocyclooctyne-N-hydroxysuccinimidyl ester (NHS-DBCO) at room temperature for 1 hour to be subjected to DBCO modification. Subsequently, rBC2LCN subjected to DBCO modification was mixed with a 10-fold molar concentration of an azinated oligonucleotide (SEQ ID NO: 8) and reacted at 4 °C overnight to obtain a nucleic acid-labeled lectin. The oligonucleotide was subjected to the azination modification at the 5′ terminal. Subsequently, the nucleic acid-labeled lectin was purified by affinity chromatography using fucose Sepharose.

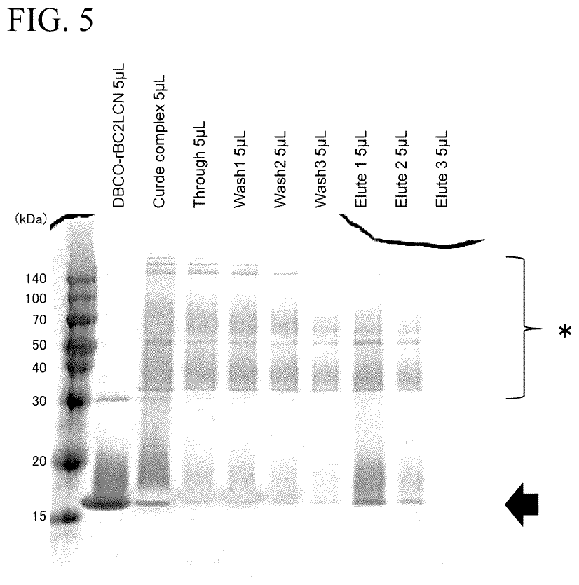

FIG. 5 is a photographic image showing the results obtained by subjecting specimens from each process of the purification of nucleic acid-labeled lectin to SDS-PAGE and subjecting them to silver staining. In FIG. 5, “DBCO-rBC2LCN” indicates rBC2LCN subjected to DBCO modification, and “Crude complex” indicates an unpurified nucleic acid-labeled lectin. In addition, “Through” indicates a specimen that has passed through the affinity column, “Wash 1”, “Wash 2”, and “Wash 3” respectively indicate the first, second, and third washing solutions, and “Elute 1”, “Elute 2”, and “Elute 3” respectively indicate the first, second, and third elution solutions. Further, “*” indicates rBC2LCN to which the oligonucleotide is bonded, and the arrow indicates rBC2LCN to which the oligonucleotide is not bonded.

As a result, rBC2LCN having an increased molecular weight due to the bonding of oligonucleotides was detected. From this result, it was confirmed that a nucleic acid-labeled lectin can be prepared by the click reaction.

Subsequently, according to the same method as in rBC2LCN, each of nucleic acid-labeled lectins was prepared, where they were obtained by bonding the oligonucleotides shown in Table 6 to a recombinant (rABA) of an ABA lectin, a recombinant (rLSLN) of an LSLN lectin, an SNA lectin (catalog number: “L-1300”, Vector Laboratories, Inc.), a GSLII lectin (catalog number: “L-1210”, Vector Laboratories, Inc.), a recombinant (rAAL) of an AAL lectin, and concanavalin A (ConA, catalog number: “300036”, SEIKAGAKU CORPORATION).

Each lectin was purified by affinity chromatography using Sepharose beads on which the sugar to which each lectin could be bound had been immobilized. Specifically, Sepharose beads on which N-acetylglucosamine had been immobilized were used for the purification of rABA, Sepharose beads on which galactose had been immobilized were used for the purification of rLSLN, Sepharose beads on which lactose had been immobilized were used for the purification of SNA, Sepharose beads on which N-acetylglucosamine had been immobilized were used for the purification of GSLII, Sepharose beads on which fucose had been immobilized were used for the purification of rAAL, and Sepharose beads on which mannose had been immobilized were used for the purification of ConA.

As a result of subjecting specimens from each process of the purification to SDS-PAGE and subjecting them to silver staining, it was confirmed that rABA, rLSLN, SNA, GSLII, rAAL, and ConA could be labeled with a nucleic acid.

### Experimental Example 5

### (Preparation of Nucleic Acid-Labeled Lectin 5)

The click reaction between an azide group and an alkyne group was used to prepare a nucleic acid-labeled lectin capable of releasing the nucleic acid upon irradiation with light. Specifically, first, a 16-fold molar concentration of an NHS-PC-DBCO ester (catalog number: “1160”, Click Chemistry Tools, LLC) was reacted with rBC2LCN at room temperature for 1 hour to subject it to PC-DBCO modification.

Subsequently, rBC2LCN subjected to PC-DBCO modification was mixed with a 10-fold molar concentration of an azinated oligonucleotide (SEQ ID NO: 8) and reacted at 4 °C overnight to obtain a nucleic acid-labeled lectin. The oligonucleotide was subjected to the azination modification at the 5′ terminal. Subsequently, the nucleic acid-labeled lectin was purified by affinity chromatography using fucose Sepharose.

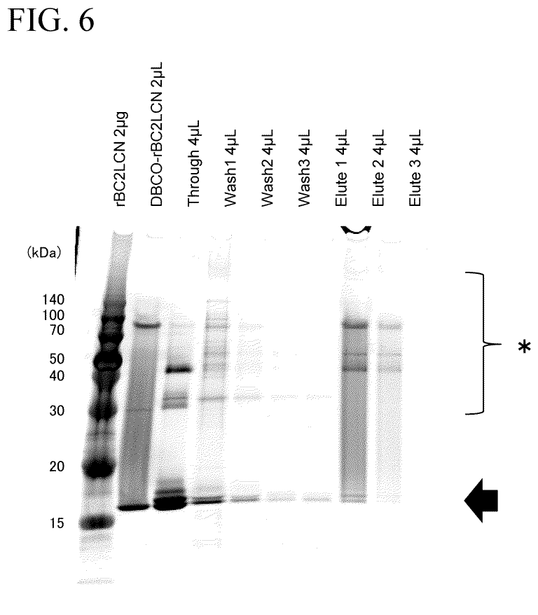

FIG. 6 is a photographic image showing the results obtained by subjecting specimens from each process of the purification of nucleic acid-labeled lectin to SDS-PAGE and subjecting them to silver staining. In FIG. 5, “PC-DBCO-rBC2LCN” indicates rBC2LCN subjected to PC-DBCO modification, “Through” indicates a specimen that has passed through the affinity column, “Wash 1”, “Wash 2”, and “Wash 3” respectively indicate the first, second, and third washing solutions, and “Elute 1”, “Elute 2”, and “Elute 3” respectively indicate the first, second, and third elution solutions. Further, “*” indicates rBC2LCN to which the oligonucleotide is bonded, and the arrow indicates rBC2LCN to which the oligonucleotide is not bonded.

As a result, rBC2LCN having an increased molecular weight due to the bonding of oligonucleotides was detected. From this result, it was confirmed that a nucleic acid-labeled lectin capable of releasing the nucleic acid upon irradiation with light can be prepared by the click reaction.

Subsequently, according to the same method as in rBC2LCN, each of nucleic acid-labeled lectins was prepared, where they were obtained by cleavably bonding the oligonucleotides shown in Table 6 to rABA, ILSLN, an SNA lectin (catalog number: “L-1300”, Vector Laboratories, Inc.), a GSLII lectin (catalog number: “L-1210”, Vector Laboratories, Inc.), rAAL, and concanavalin A (ConA, catalog number: “300036”, SEIKAGAKU CORPORATION) upon irradiation with light.

Each lectin was purified by affinity chromatography using Sepharose beads on which the sugar to which each lectin could be bound had been immobilized. As a result of subjecting specimens from each process of the purification to SDS-PAGE and subjecting them to silver staining, it was confirmed that each of the nucleotides could be cleavably bonded to rABA, ILSLN, SNA, GSLII, rAAL, and ConA upon irradiation with light.

### Experimental Example 6

### (Examination of Reaction Conditions Between Nucleic Acid-Labeled Lectin and Cell)

The reaction conditions between the nucleic acid-labeled lectin and cells were examined. Specifically, the nucleic acid-labeled lectin and cells were reacted and compared under the conditions of method 1 and method 2 below.

### <<Method 1>>

As the nucleic acid-labeled lectin, a nucleic acid-labeled rBC2LCN lectin prepared in the same manner as in Experimental Example 4 was used. In addition, as the cell, a MIAPaCa-2 cell, a BxPC-3 cell, and a Capan-1 cell, all of which are cells derived from human pancreatic cancer, were used.

First, 1×10⁵ cells of each of the above cells was suspended in each 100 μL of phosphate-buffered saline (PBS) containing 1% by mass of bovine serum albumin (BSA) (hereinafter, may be referred to as “BSA-PBS”). Subsequently, 100 ng of the nucleic acid-labeled rBC2LCN lectin was added to each cell suspension and reacted at 4 °C for 1 hour. Subsequently, each cell was washed 3 times with 1 mL of BSA-PBS and then suspended in 200 μL of BSA-PBS.

Subsequently, 1×10⁴ live cells were placed in a new tube, centrifuged to remove the supernatant, 100 μL of 0.2 M fucose was added thereto and reacted at 4 °C for 30 minutes to release the nucleic acid-labeled rBC2LCN lectin from the cells. Subsequently, the supernatant was recovered by centrifugation at 15,000 rpm for 10 minutes, and the oligonucleotide bonded to the rBC2LCN lectin was quantified by real-time quantitative PCR.

For comparison, each cell was stained with an rBC2LCN lectin labeled with R-phycoerythrin, and the flow cytometric analysis was carried out.

### <<Method 2>>

Method 2 was mainly different from method 1 in that the reaction between the nucleic acid-labeled lectin and cells was carried out in an OptiMEM culture medium rather than in BSA-PBS.

As the nucleic acid-labeled lectin and cells, the same ones as in Method 1 were used. First, 1×10⁵ cells of each of the above cells were suspended in 100 μL of an OptiMEM culture medium (Thermo Fisher Scientific, Inc.). Subsequently, 100 ng of the nucleic acid-labeled rBC2LCN lectin was added to each cell suspension and reacted at 4 °C for 1 hour.

Subsequently, each cell was washed 3 times with 1 mL of BSA-PBS and then suspended in 200 μL of PBS.

Subsequently, 1×10⁴ live cells were placed in a new tube, centrifuged to remove the supernatant, 100 μL of 0.2 M fucose was added thereto and reacted at 4 °C for 30 minutes to release the nucleic acid-labeled rBC2LCN lectin from the cells.

Subsequently, the supernatant was recovered by centrifugation at 15,000 rpm for 10 minutes, and the oligonucleotide bonded to the rBC2LCN lectin was quantified by real-time quantitative PCR.

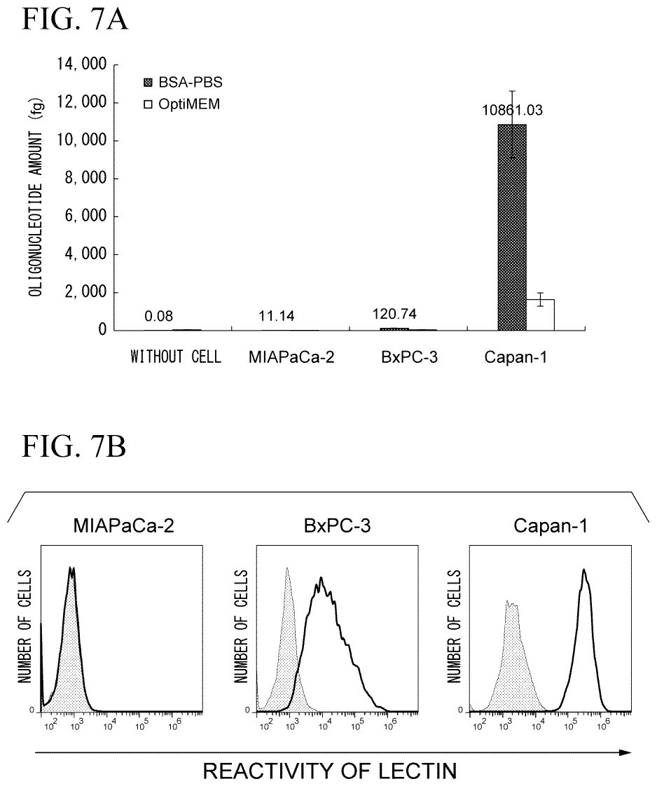

FIG. 7A is a graph showing the results of real-time quantitative PCR. In FIG. 7A, “BSA-PBS” indicates the result of method 1, and “OptiMEM” indicates the result of method 2. As a result, it was revealed that the binding between the cell and the lectin can be detected with higher sensitivity in method 1.

In addition, FIG. 7B is graphs showing the results of flow cytometric analysis. As a result, it was revealed that the analysis result from the real-time quantitative PCR shown in FIG. 7A is in agreement with the result of flow cytometric analysis, and the rBC2LCN lectin exhibits the highest reactivity to the Capan-1 cell, also reacts to the BxPC-3 cell, but does not react to the MIAPaCa-2 cell.

### Experimental Example 7

(Examination of Nucleic Acid Release Conditions Upon Irradiation with Light)

The conditions for releasing the nucleic acid from the nucleic acid-labeled lectin capable of releasing the nucleic acid upon irradiation with light were examined. As the nucleic acid-labeled lectin, a nucleic acid-labeled rBC2LCN lectin prepared in the same manner as in Experimental Example 5 was used. In addition, as the cell, a Capan-1 cell, which is a cell derived from human pancreatic cancer, was used.

First, 1×10⁵ cells were suspended in 100 μL of BSA-PBS. Subsequently, 100 ng of the nucleic acid-labeled rBC2LCN lectin was added to the cell suspension and reacted at 4 °C for 1 hour. Subsequently, the cells were washed 3 times with 1 mL of BSA-PBS and then suspended in 200 μL of BSA-PBS.

Subsequently, each aliquot of 1×10⁴ cells was placed in a new tube, and an ultraviolet (UV) irradiation device (catalog number: “95-0042-14”, Funakoshi Co., Ltd.) was used to carry out irradiation with ultraviolet rays having a peak wavelength at a wavelength of 365 nm at 15 W for 5 minutes, 10 minutes, or 15 minutes, thereby releasing the oligonucleotide. Subsequently, the supernatant was recovered by centrifugation at 15,000 rpm for 10 minutes, and the oligonucleotide bonded to the rBC2LCN lectin was quantified by real-time quantitative PCR.

For comparison, the oligonucleotide bonded to the rBC2LCN lectin was quantified by real-time quantitative PCR on a specimen of a supernatant recovered by immediate centrifugation without ultraviolet irradiation, a specimen of a supernatant recovered by centrifugation after allowing to stand at room temperature for 15 minutes without ultraviolet irradiation, and a specimen of a supernatant recovered by centrifugation after adding 100 μL of 0.2 M fucose to allow it to react at 4 °C for 15 minutes and releasing the nucleic acid-labeled rBC2LCN lectin from the cells.

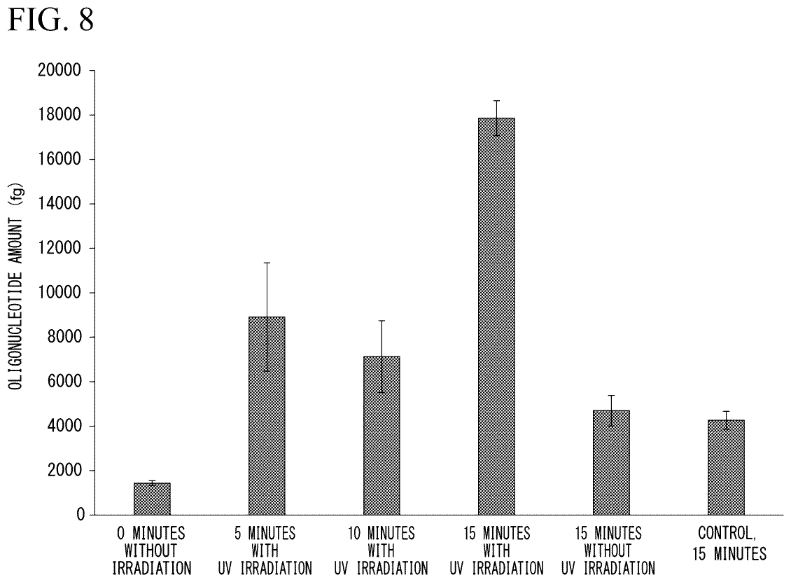

FIG. 8 is a graph showing the results of real-time quantitative PCR. In FIG. 8, “0 minutes without irradiation” indicates the result of a specimen of a supernatant recovered by centrifuging at 15,000 rpm for 10 minutes without ultraviolet irradiation, “15 minutes without irradiation” indicates the result of a specimen of a supernatant recovered by centrifuging at 15,000 rpm for 10 minutes after allowing to stand at room temperature for 15 minutes without ultraviolet irradiation, and “Control, 15 minutes” indicates the result of a specimen of a supernatant recovered by centrifuging at 15,000 rpm for 10 minutes after adding 100 μL of 0.2 M fucose to allow it to react at 4 °C for 15 minutes to release the nucleic acid-labeled rBC2LCN lectin from the cells.

As a result, it was revealed that the largest number of oligonucleotides can be released in a case of being irradiated with ultraviolet rays for 15 minutes.

### Experimental Example 8

### (Examination of Reactivity of Nucleic Acid-Labeled Lectin 1)

It was examined whether or not there was a difference in the reactivity between an ordinary lectin not labeled with a nucleic acid and the nucleic acid-labeled lectin.

Specifically, each of the ordinary lectin (the unlabeled lectin) and the nucleic acid-labeled lectin was labeled with fluorescein isothiocyanate (FITC) and reacted with cells, and then flow cytometric analysis was carried out.

As the unlabeled lectin, GSLII, rABA, rBC2LCN, rLSLN, and SNA were used. As the nucleic acid-labeled lectin, nucleic acid-labeled bodies of the same lectins (GSLII, rABA, rBC2LCN, rLSLN, and SNA) as described above, prepared in the same manner as in Experimental Example 4, were used. As the cell, a SUIT-2 cell, an AsPC-1 cell, a BxPC-3 cell, a MIAPaCa-2 cell, and a Capan-1 cell, all of which are human pancreatic cancer-derived cells, were used.

First, each aliquot of 1×10⁵ cells of each cell was suspended in 100 μL of BSA-PBS and dispensed into a tube. Subsequently, 100 ng of each of the unlabeled lectins (GSLII, rABA, rBC2LCN, rLSLN, and SNA) or nucleic acid-labeled lectins (GSLII, rABA, rBC2LCN, ILSLN, and SNA) was added to each cell suspension and reacted at 4 °C for 1 hour. Subsequently, each cell was washed 3 times with 1 mL of BSA-PBS and then suspended in 500 μL of BSA-PBS.

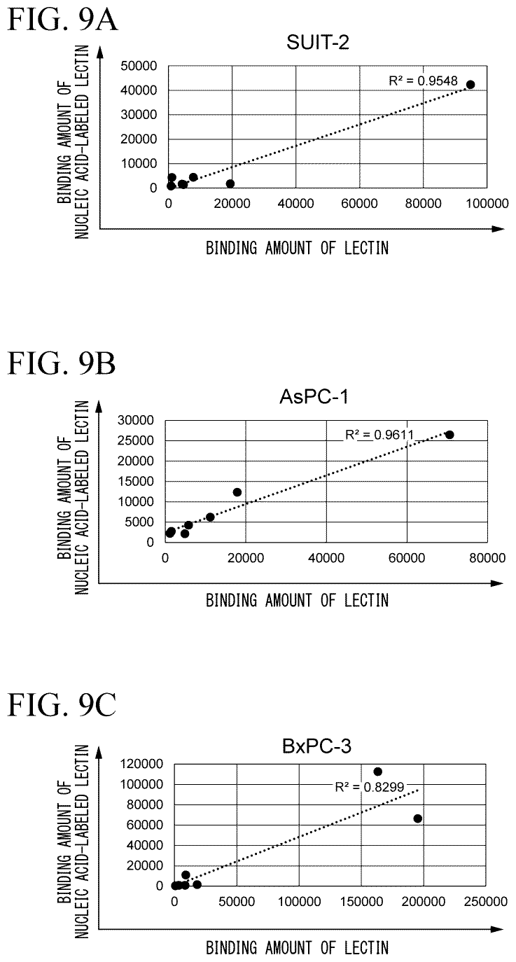

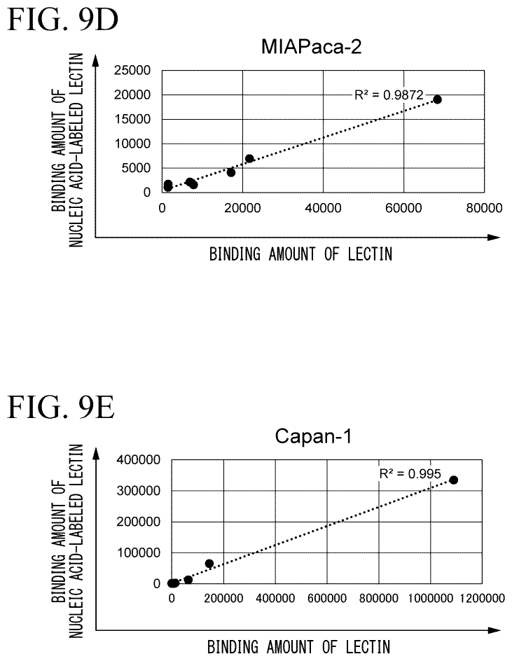

Subsequently, each cell was subjected to flow cytometric analysis, the amount of each lectin bound to the cell was measured, and the correlation of the reactivity between the unlabeled body and the nucleic acid labeled body was investigated for each lectin. FIG. 9A to FIG. 9E are graphs showing the results of flow cytometric analysis. FIG. 9A is the result of the SUIT-2 cell, FIG. 9B is the result of the AsPC-1 cell, FIG. 9C is the result of the BxPC-3 cell, FIG. 9D is the result of the MIAPaCa-2 cell, and FIG. 9E is the result of the Capan-1 cell. Further, in FIG. 9A to FIG. 9E, the lateral axis indicates the binding amount (the average fluorescence intensity) of the unlabeled lectins (GSLII, rABA, rBC2LCN, rLSLN, and SNA), and the vertical axis indicates the binding amount (the average fluorescence intensity) of the nucleic acid-labeled lectins (GSLII, rABA, rBC2LCN, rLSLN, and SNA).

As a result, it was revealed that the reactivity of the unlabeled lectin and the reactivity of the nucleic acid-labeled lectin to each cell each show a high correlation. From this result, it was confirmed that the reactivity of the lectin does not change even in a case where the lectin is labeled with a nucleic acid.

### Experimental Example 9

### (Examination of Reactivity of Nucleic Acid-Labeled Lectin 2)

The nucleic acid-labeled lectin was reacted with cells, and the binding amount thereof was measured and compared by flow cytometric analysis and real-time quantitative PCR analysis.

As the nucleic acid-labeled lectin, nucleic acid-labeled lectins (GSLII, rABA, rBC2LCN, rLSLN, and SNA) prepared in the same manner as in Experimental Example 5 and capable of releasing the nucleic acid upon irradiation with light were used. In addition, as the cell, a Capan-1 cell, which is a cell derived from human pancreatic cancer, was used.

First, each aliquot of 1×10⁵ cells was suspended in 100 μL of BSA-PBS and dispensed into a tube. Subsequently, 100 ng of the nucleic acid-labeled lectins (GSLII, rABA, rBC2LCN, rLSLN, and SNA) were added to each cell suspension and reacted at 4 °C for 1 hour.

Subsequently, each cell was washed 3 times with 1 mL of BSA-PBS and then suspended in 500 μL of BSA-PBS.

Subsequently, some cells were subjected to flow cytometric analysis, and the remainder were subjected to real-time quantitative PCR to measure the amount of lectin bound to the cell. Real-time quantitative PCR was carried out as follows. First, an ultraviolet (UV) irradiation device (catalog number: “95-0042-14”, Funakoshi Co., Ltd.) was used to irradiate a part of cells with ultraviolet rays having a peak wavelength at a wavelength of 365 nm at 15 W for 15 minutes, thereby releasing the oligonucleotide. Subsequently, the supernatant was recovered by centrifugation at 15,000 rpm for 10 minutes, and the oligonucleotide released was quantified by real-time quantitative PCR.

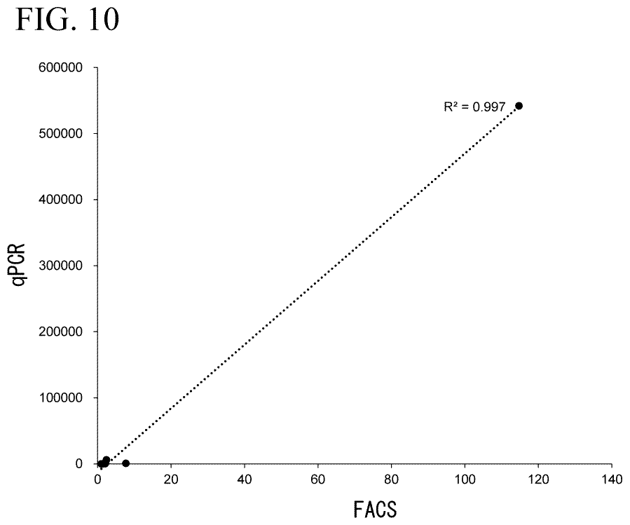

FIG. 10 is a graph showing the results of flow cytometric analysis and real-time quantitative PCR. In FIG. 10, “qPCR” indicates the result of real-time quantitative PCR (the oligonucleotide amount), and “FACS” indicates the result of flow cytometric analysis (the average fluorescence intensity).

As a result, it was revealed that the result of flow cytometric analysis and the result of real-time quantitative PCR show a high correlation. From this result, it was revealed that the same result as the flow cytometric analysis can be obtained by real-time quantitative PCR using a nucleic acid-labeled lectin.

### Experimental Example 10

### (Examination of Reactivity of Nucleic Acid-Labeled Lectin 3)

A plurality of kinds of lectins were reacted with cells, and the binding of the lectins was analyzed by flow cytometric analysis and real-time quantitative PCR. Specifically, GSLII, rABA, BC2LCN, rLSLN, and SNA, each labeled with fluorescein isothiocyanate (FITC), were each reacted with the Capan-1 cell, and the flow cytometric analysis was carried out.

First, each aliquot of 1×10⁵ cells of the Capan-1 cell was suspended in 100 μL of BSA-PBS and dispensed into a tube. Subsequently, 100 ng of each FITC-labeled lectin was added to each cell suspension and reacted at 4 °C for 1 hour. Subsequently, each cell was washed 3 times with 1 mL of BSA-PBS and then suspended in 200 μL of BSA-PBS. Subsequently, each cell was subjected to flow cytometric analysis, and the amount of each lectin bound to the cell was measured.

In addition, each of GSLII, rABA, rBC2LCN, rLSLN, and SNA, which were subjected to nucleic acid labeling in the same manner as in Experimental Example 5, was reacted with the Capan-1 cell, and the bound lectin was measured by real-time quantitative PCR.

Specifically, each aliquot of 1×10⁵ cells of the Capan-1 cell was suspended in 100 μL of BSA-PBS and dispensed into a tube. Subsequently, 100 ng of each nucleic acid-labeled lectin was added to each cell suspension and reacted at 4 °C for 1 hour. Subsequently, each cell was washed 3 times with 1 mL of BSA-PBS and then suspended in 500 μL of BSA-PBS.

Subsequently, an ultraviolet (UV) irradiation device (catalog number: “95-0042-14”, Funakoshi Co., Ltd.) was used to irradiate each cell with ultraviolet rays having a peak wavelength at a wavelength of 365 nm at 15 W for 15 minutes, thereby releasing the oligonucleotide. Subsequently, the supernatant was recovered by centrifugation at 15,000 rpm for 10 minutes, and each oligonucleotide released was quantified by real-time quantitative PCR.

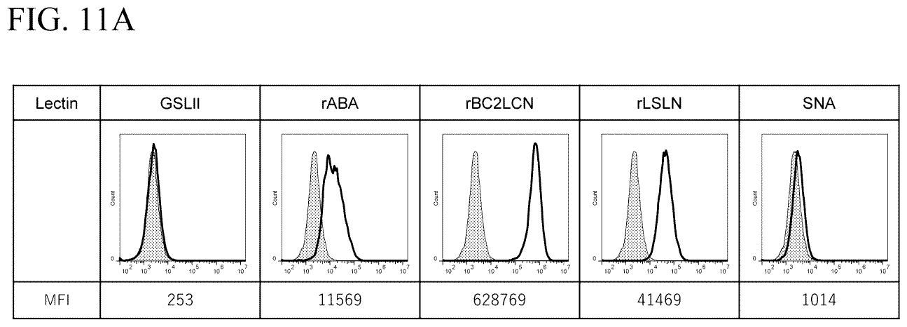

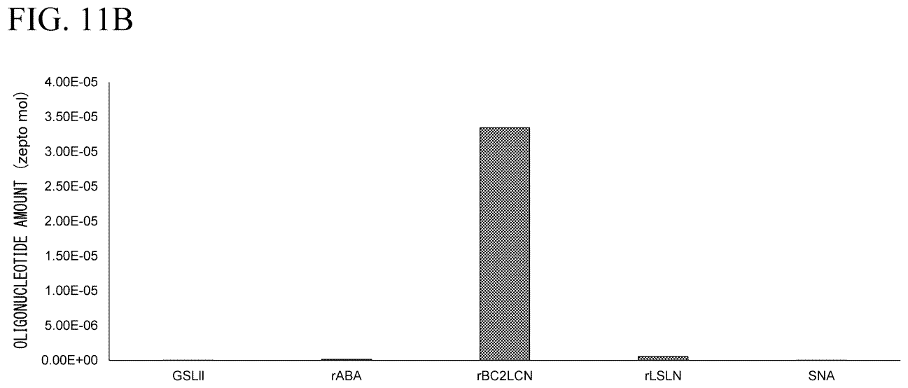

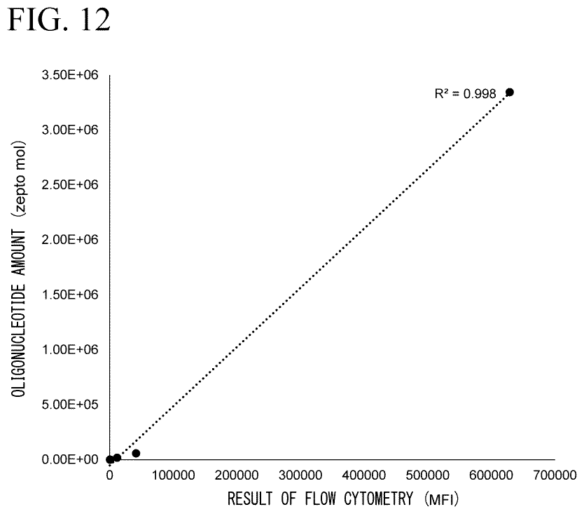

FIG. 11A is graphs showing the results of flow cytometric analysis. In FIG. 11A, “MFI” indicates the average value of the measured fluorescence intensities. In addition, FIG. 11B is a graph showing the results of real-time quantitative PCR. In addition, FIG. 12 is a graph showing the results of flow cytometric analysis on the lateral axis and the results of real-time quantitative PCR on the vertical axis based on the results of FIG. 11A and FIG. 11B.

As a result, it was confirmed that the results of flow cytometric analysis and the result of real-time quantitative PCR show a high correlation. These results further support that the binding of the lectin to the cell can be analyzed by reacting the nucleic acid-labeled lectin with cells and detecting the nucleic acid bonded to the nucleic acid-labeled lectin.

### Experimental Example 11

### (Examination of Reactivity of Nucleic Acid-Labeled Lectin 4)

The nucleic acid-labeled lectin was reacted with serially diluted cells and the binding amount thereof was measured by real-time quantitative PCR analysis.

As the nucleic acid-labeled lectin, a nucleic acid-labeled lectin rBC2LCN lectin prepared in the same manner as in Experimental Example 5 and capable of releasing the nucleic acid upon irradiation with light was used. In addition, as the cell, a Capan-1 cell, which is a cell derived from human pancreatic cancer, was used.

First, 10,000 cells, 1,000, 100 cells, 10 cells, 1 cell, and 0 cells of the Capan-1 cell were each suspended in 100 μL of BSA-PBS and placed in a tube. Subsequently, 100 ng of the nucleic acid-labeled rBC2LCN lectin was added to each cell suspension and reacted at 4 °C for 1 hour. Subsequently, each cell was washed 3 times with 1 mL of BSA-PBS and then suspended in 200 μL of BSA-PBS.

Subsequently, an ultraviolet (UV) irradiation device (catalog number: “95-0042-14”, Funakoshi Co., Ltd.) was used to irradiate each cell with ultraviolet rays having a peak wavelength at a wavelength of 365 nm at 15 W for 15 minutes, thereby releasing the oligonucleotide. Subsequently, the supernatant was recovered by centrifugation at 15,000 rpm for 10 minutes, and each oligonucleotide released was quantified by real-time quantitative PCR.

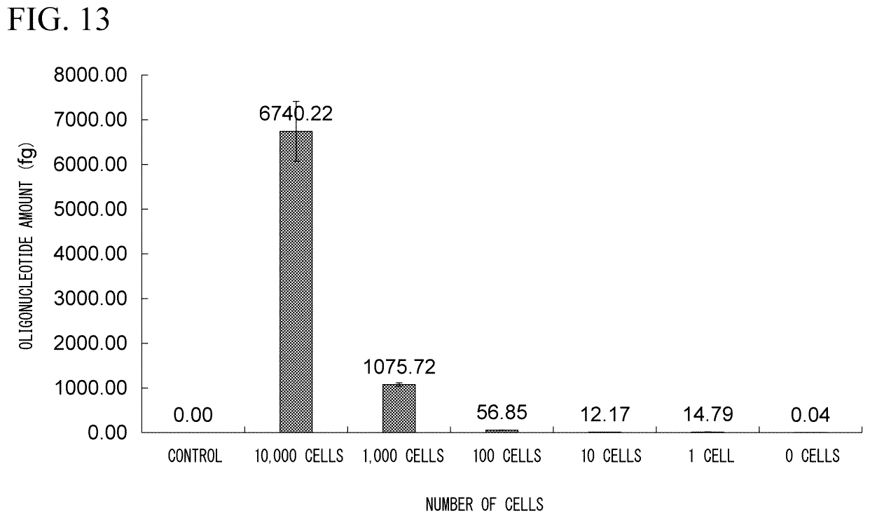

FIG. 13 is a graph showing the results of real-time quantitative PCR. In FIG. 13, “Control” is the result of a specimen obtained by undergoing a series of reactions without adding the nucleic acid-labeled rBC2LCN lectin. As a result, it was revealed that the binding of the lectin to even a single cell can be analyzed by reacting the nucleic acid-labeled lectin with cells and detecting the nucleic acid bonded to the nucleic acid-labeled lectin.

### Experimental Example 12

### (Examination of Reactivity of Nucleic Acid-Labeled Lectin 5)

A mixture of a plurality of kinds of nucleic acid-labeled lectins was reacted with 100,000 cells, and the nucleic acid bound to 10,000 cells among them was analyzed using a next generation sequencer. Nucleic acid-labeled lectins (SNA, ConA, GSLII, rBC2LCN, rAAL, rABA, ILSLN, rPSL1a, rDiscoidin I, IF17AG, rPVL, rCGL2, rPAIL, rPPL, rRSIIL, rCNL, WFA, HPA, SSA, rOrysata, rPALa, rDiscoidin II, CSA, rGRFT, rSRL, rAOL, rBanana, rBC2LA, rC14, rCalsepa, rGal3C, rGC2, rMalectin, rMOA, rPTL, rRSL, TJAI, TJAII, and, UEAI) prepared in the same manner as in Experimental Example 4 were used as the nucleic acid-labeled lectin, and a podocalyxin (PODXL) antibody (manufactured by R&D Systems, Inc.) was used. In addition, as the cell, a Capan-1 cell, a BxPC-3 cell, a PANC-1 cell, and an AsPC1 cell, which are cells derived from human pancreatic cancer, as well as a CHO cell and Lec1, Lec2, and Lec8, which are mutant cells of the CHO cell, were used.

First, each aliquot of 100,000 cells of each cell was suspended in 100 μL of BSA-PBS and dispensed into a tube. Subsequently, 50 ng of each nucleic acid-labeled lectin was added to each cell suspension and reacted at 4 °C for 1 hour. Subsequently, each cell was washed 3 times with 1 mL of BSA-PBS and then suspended in 200 μL of BSA-PBS. Subsequently, the number of cells was measured, and 10,000 were transferred to a new tube. Subsequently, the supernatant was removed by centrifugation, and then the cells were suspended in 100 μL PBS. Subsequently, an ultraviolet (UV) irradiation device (catalog number: “95-0042-14”, Funakoshi Co., Ltd.) was used to carry out irradiation with ultraviolet rays having a peak wavelength at a wavelength of 365 nm at 15 W for 15 minutes and to release the oligonucleotide, thereby recovering the supernatant after centrifugation. Subsequently, a PCR reaction was carried out using this as a template, and the analysis was carried out using a next generation sequencer.

The following operations were carried out in a case of carrying out the sequencing with the next generation sequencer. PCR was carried out with an 15 index primer (SEQ ID NO: 75) and an I7 index primer (SEQ ID NO: 76), the amplified PCR product was purified and concentrated, and then the degree of purification and the concentration thereof were checked with a microchip electrophoresis device for DNA/RNA analysis (Shimadzu Corporation). Subsequently, the base sequence of the nucleic acid labeled to the nucleic acid-labeled lectin bound to each cell was sequenced using a next generation sequencer (product name: “MiSeq”, Illumina, Inc.), and the number of reads of the detected base sequence was totaled. Further, “nnnnnnnn” in SEQ ID NOs: 75 and 76 (here, “n” indicates “a”, “t”, “g”, or “c”) has a different sequence for each cell, which is used for identification of cells.

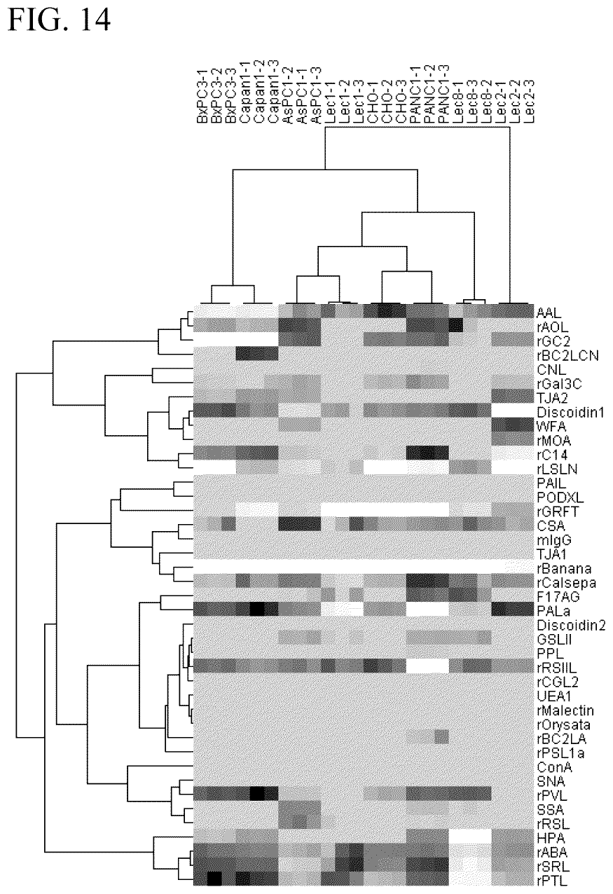

FIG. 14 is a diagram showing the results of cluster analysis using the number of reads of the base sequence of the nucleic acid labeled to each lectin.

As a result, it was revealed that the binding of the lectin to the cell can be analyzed by reacting the nucleic acid-labeled lectin with 10,000 cells and sequencing the nucleic acid bonded to the nucleic acid-labeled lectin with a next generation sequencer. Further, it was revealed that the pattern of the reaction with the lectin was different in each cell.

### Experimental Example 13

### (Examination of Reactivity of Nucleic Acid-Labeled Lectin 6)

Each mixture of a plurality of kinds of nucleic acid-labeled lectins was reacted with 10,000 cells and a single cell, and the binding thereof was analyzed using a next generation sequencer. Nucleic acid-labeled lectins (SNA, ConA, GSLII, rBC2LCN, rAAL, rABA, ILSLN, rPSL1a, rDiscoidin I, rF17AG, rPVL, rCGL2, rPAIL, rPPL, rRSIIL, rCNL, WFA, HPA, SSA, rOrysata, rPALa, rDiscoidin II, CSA, rGRFT, rSRL, rAOL, rBanana, rBC2LA, rC14, rCalsepa, rGal3C, rGC2, rMalectin, rMOA, rPTL, rRSL, TJAI, TJAII, and, UEAI) prepared in the same manner as in Example 4 were used as the nucleic acid-labeled lectin, and a podocalyxin (PODXL) antibody was used. In addition, as the cell, 201B7, which is a human iPS cell line, was used.

First, each aliquot of 100,000 cells of the human iPS cell was suspended in 100 μL of BSA-PBS and dispensed into a tube. Subsequently, 0.5 ng of each nucleic acid-labeled lectin was added to each cell suspension and reacted at 4 °C for 1 hour. Subsequently, the cells were washed 3 times with 1 mL of BSA-PBS and then suspended in 200 μL of BSA-PBS. Subsequently, 10,000 cells and a single cell were dispensed, the base sequence of the nucleic acid labeled to the nucleic acid-labeled lectin bound to each cell was sequenced using a next generation sequencer (product name: “MiSeq”, Illumina, Inc.), and the number of reads of the detected base sequence was totaled.

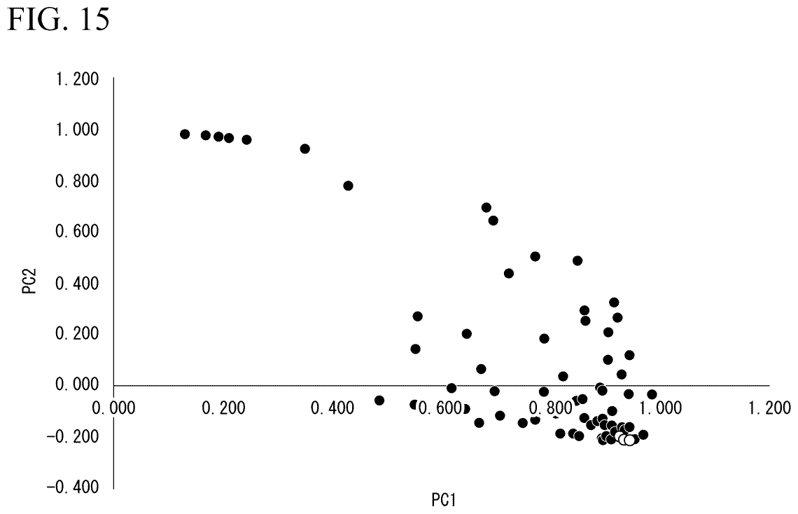

FIG. 15 is a graph showing the results obtained by subjecting the obtained number of reads to the main component analysis. In FIG. 15, “PC1” indicates a Principal component 1 (a main component 1), and “PC2” indicates a Principal component 2 (a main component 2). In the graph, black circles indicate the results of the analysis at a single cell level, and white circles indicate the results of the analysis at the level of 10,000 cells.

As a result, it was revealed that the binding of the lectin to the cell can be analyzed at a single cell level by reacting a mixture of the nucleic acid-labeled lectins with cells and sequencing the nucleic acid bonded to the nucleic acid-labeled lectin with a next generation sequencer. In addition, it was revealed that although the analysis result at a single cell level is similar to the analysis result of 10,000 cells, this cell population has considerably heterogeneous glycans.

### Experimental Example 14

### (Examination of Reactivity of Nucleic Acid-Labeled Lectin 7)

Each mixture of a plurality of kinds of nucleic acid-labeled lectins was reacted with 10,000 cells of the microorganism, and the binding thereof was analyzed using a next generation sequencer. Nucleic acid-labeled lectins (rBC2LCN, rAAL, rABA, ILSLN, rPSL1a, rDiscoidin I, rF17AG, rPVL, rCGL2, rPAIL, rPPL, rRSIIL, rCNL, WFA, HPA, SSA, rOrysata, rPALa, and rDiscoidin II) prepared in the same manner as in Example 4 were used as the nucleic acid-labeled lectin. As the microorganism, Escherichia coli, Deinococcus radiodurans, and budding yeast (Saccharomyces cerevisiae) were used.

First, each aliquot of 1,000,000 cells of each microorganism was suspended in 100 μL of BSA-PBS and dispensed into a tube. Subsequently, 0.5 ng of each nucleic acid-labeled lectin was added to each cell suspension and reacted at 4 °C for 1 hour. Subsequently, each microorganism was washed 3 times with 1 mL of BSA-PBS and then suspended in 200 μL of BSA-PBS.

Subsequently, 100,000 microorganisms of each microorganism were dispensed, the base sequence of the nucleic acid labeled to the nucleic acid-labeled lectin bound to each microorganism was sequenced using a next generation sequencer (product name: “MiSeq”, Illumina, Inc.), and the number of reads of the detected base sequence was totaled. The sequencing was independently carried out 3 times for each microorganism.

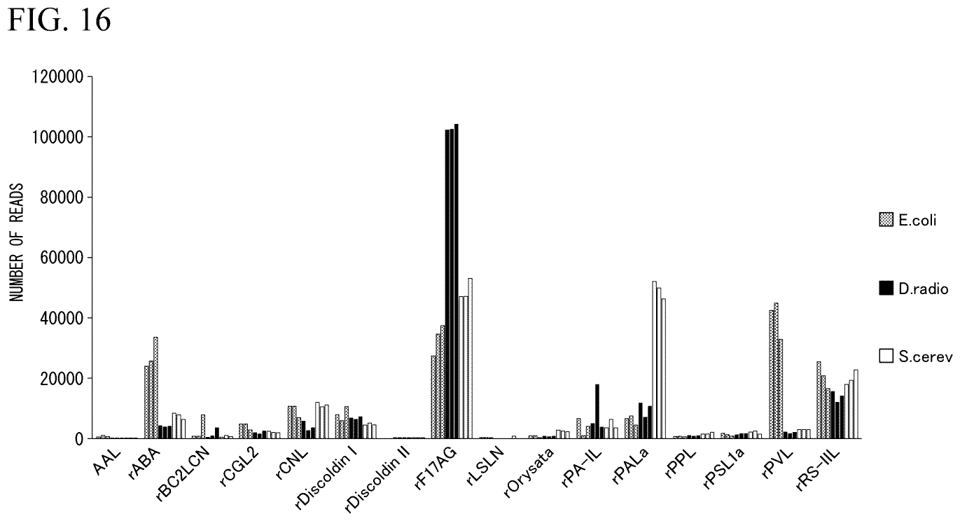

FIG. 16 is a graph showing the number of reads of the base sequence of the nucleic acid labeled to each lectin. In FIG. 16, “E. coli” indicates Escherichia coli, “D. radio” indicates Deinococcus radiodurans, and “S. cerev” indicates Saccharomyces cerevisiae. As a result, it was revealed that the binding of the lectin to the microorganism can be analyzed at the level of a single microorganism by reacting the nucleic acid-labeled lectin with cells of the microorganism and sequencing the nucleic acid bonded to the nucleic acid-labeled lectin with a next generation sequencer.

### Experimental Example 15

### (Examination of Reactivity of Nucleic Acid-Labeled Lectin 8)

A human iPS cell 201B7 line was differentiated into ectoderm by using a commercially available kit (product name: “STEMdiff SMADi Neural Induction Kit, 2 pack”, catalog number: “#08582”, STEMCELL Technologies), and the cell was recovered on the 4th day and the 11th day. Subsequently, each plurality of kinds of nucleic acid-labeled lectins was subjected to a reaction, and the binding thereof was analyzed using a next generation sequencer.

Nucleic acid-labeled lectins (SNA, ConA, GSLII, rBC2LCN, rAAL, rABA, ILSLN, rPSL1a, rDiscoidin I, rF17AG, rPVL, rCGL2, rPAIL, rPPL, rRSIIL, rCNL, WFA, HPA, SSA, rOrysata, rPALa, rDiscoidin II, CSA, rGRFT, rSRL, rAOL, rBanana, rBC2LA, rC14, rCalsepa, rGal3C, rGC2, rMalectin, rMOA, rPTL, rRSL, TJAI, TJAII, and, UEAI) prepared in the same manner as in Example 4 were used as the nucleic acid-labeled lectin, and a podocalyxin (PODXL) antibody was used.

First, each aliquot of 100,000 cells of each cell was suspended in 100 μL of BSA-PBS and dispensed into a tube. Subsequently, 0.5 ng of each nucleic acid-labeled lectin was added to each cell suspension and reacted at 4 °C for 1 hour. Subsequently, the cells were washed 3 times with 1 mL of BSA-PBS and then suspended in 200 μL of BSA-PBS.

Subsequently, 10,000 cells and a single cell were dispensed, the base sequence of the nucleic acid labeled to the nucleic acid-labeled lectin bound to each cell was sequenced using a next generation sequencer (product name: “MiSeq”, Illumina, Inc.), and the number of reads of the detected base sequence was totaled.

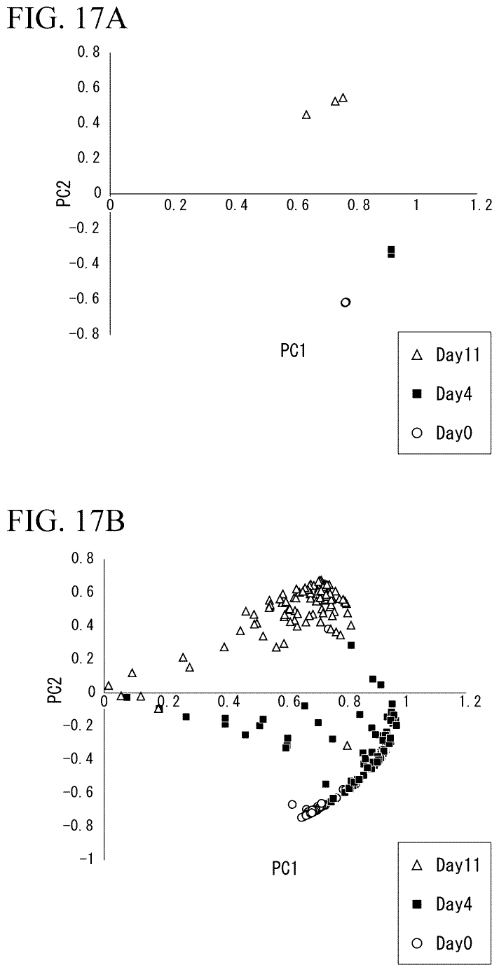

FIG. 17A and FIG. 17B are graphs showing the results obtained by subjecting the obtained number of reads to the main component analysis. FIG. 17A shows the results of the analysis at the level of 10,000 cells (the average value, n=3). Further, FIG. 17B shows the results of the analysis at a single cell level (n=3). In FIG. 17A and FIG. 17B, “PC1” indicates a Principal component 1 (a main component 1), and “PC2” indicates a Principal component 2 (a main component 2). In addition, “Day 0” indicates that it is the result of the iPS cell before differentiation induction, “Day 4” indicates that it is the result of the iPS cell on the 4th day after differentiation induction, and “Day 7” indicates that it is the result of the iPS cell on the 7th day after differentiation induction.

As a result, it was revealed that the binding of the lectin to the cell can be analyzed at a single cell level by reacting a mixture of the nucleic acid-labeled lectins with cells and sequencing the nucleic acid bonded to the nucleic acid-labeled lectin with a next generation sequencer. In addition, it was revealed that although the analysis result at a single cell level is similar to the analysis result of 10,000 cells, this cell population has considerably heterogeneous glycans.

In addition, from the analysis results at the level of 10,000 cells, it was revealed that the glycan structure changes with the elapse of time after the induction of differentiation. In addition, from the analysis results at a single cell level, it was revealed that although the iPS cell is a relatively homogeneous cell population, it becomes a cell population having various glycans after the induction of differentiation.

This result further supports that the glycan of individual cells constituting a cell population can be analyzed at a single cell level.

### Experimental Example 16

### (Analysis of a Cell Surface Glycan and RNA in Cell at a Single Cell Level 1)

Each aliquot of 100,000 cells of a human iPS cell 201B7 line was suspended in 100 μL of BSA-PBS and dispensed into a tube. Subsequently, 0.5 ng of each nucleic acid-labeled lectin was added to each cell suspension and reacted at 4 °C for 1 hour. Subsequently, the cells were washed 3 times with 1 mL of BSA-PBS and then suspended in 200 μL of BSA-PBS.

Subsequently, an ultraviolet (UV) irradiation device (catalog number: “95-0042-14”, Funakoshi Co., Ltd.) was used to carry out irradiation with ultraviolet rays having a peak wavelength at a wavelength of 365 nm at 15 W for 15 minutes and to release the oligonucleotide from the nucleic acid-labeled lectin, thereby recovering the supernatant after centrifugation. Subsequently, a PCR reaction was carried out using this as a template, and the analysis was carried out using a next generation sequencer.

In addition, a cDNA library was prepared from the total RNA of a single cell precipitated after centrifugation by using GenNext® RamDA-seq™ Single Cell Kit (Toyobo Co., Ltd.).

Subsequently, the sample was treated using Nextera XT DNA Sample Preparation Kit (Illumina, Inc.) and then sequenced using a next generation sequencer (product name: “MiSeq”, Illumina, Inc.), and the number of reads of the detected base sequence was totaled.

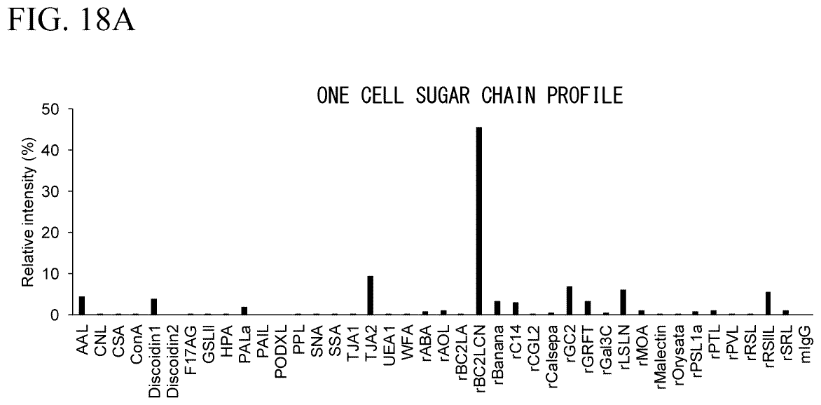

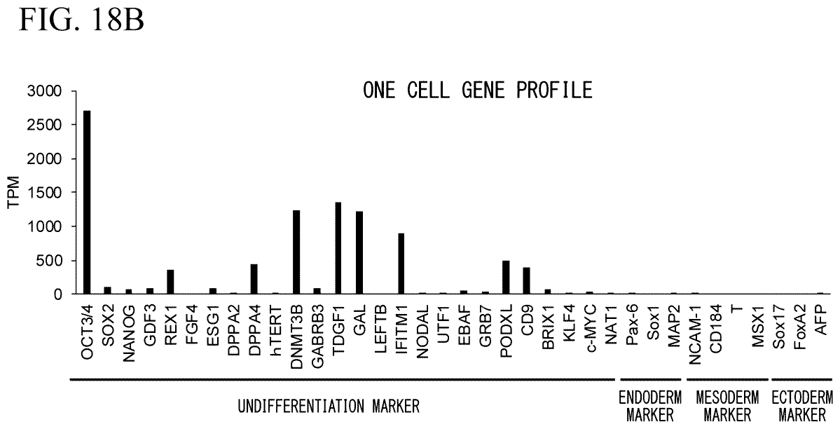

FIG. 18A and FIG. 18B are graphs showing representative results obtained by analyzing a cell surface glycan and RNA in cells at a single cell level. FIG. 18A is the results of the glycan profile, and FIG. 18B is the results of the gene profile. In FIG. 18A, the vertical axis indicates the signal intensity (the relative value), and the lateral axis indicates the kind of lectin. Further, in FIG. 18B, the vertical axis indicates transcripts per million (TPM), and the lateral axis indicates the gene name of each marker gene of the undifferentiation marker, endoderm marker, mesoderm marker, and ectoderm marker.

As a result, it was revealed that RNA information as well as glycan information on the cell surface can be obtained from a single cell.

### Experimental Example 17

### (Analysis of a Cell Surface Glycan and RNA in Cell at a Single Cell Level 2)

The human iPS cell 201B7 line was differentiated into ectoderm (nerve), and the cell surface glycan on the 0th day and 11th day and the RNA in the cell were analyzed at a single cell level.

Specifically, first, the human iPS cell 201B7 line was differentiated into ectoderm (nerve) by using a commercially available kit (product name: “STEMdiff SMADi Neural Induction Kit, 2 pack”, catalog number: “#08582”, STEMCELL Technologies), and the cell was recovered on the 0th day and the 11th day.

Subsequently, each plurality of kinds of nucleic acid-labeled lectins was reacted with each recovered cell, and the binding thereof was analyzed using a next generation sequencer. Nucleic acid-labeled lectins (SNA, ConA, GSLII, rBC2LCN, rAAL, rABA, ILSLN, rPSL1a, rDiscoidin I, rF17AG, rPVL, rCGL2, rPAIL, rPPL, rRSIIL, rCNL, WFA, HPA, SSA, rOrysata, rPALa, rDiscoidin II, CSA, rGRFT, rSRL, rAOL, rBanana, rBC2LA, rC14, rCalsepa, rGal3C, rGC2, rMalectin, rMOA, rPTL, rRSL, TJAI, TJAII, and, UEAI) prepared in the same manner as in Example 4 were used as the nucleic acid-labeled lectin, and a podocalyxin (PODXL) antibody was used.

Each aliquot of 100,000 cells of each cell was suspended in 100 μL of BSA-PBS and dispensed into a tube. Subsequently, 0.5 ng of each nucleic acid-labeled lectin was added to each cell suspension and reacted at 4 °C for 1 hour. Subsequently, the cells were washed 3 times with 1 mL of BSA-PBS and then suspended in 200 μL of BSA-PBS.

Subsequently, cells were dispensed to a single cell, and an ultraviolet (UV) irradiation device (catalog number: “95-0042-14”, Funakoshi Co., Ltd.) was used to carry out irradiation with ultraviolet rays having a peak wavelength at a wavelength of 365 nm at 15 W for 15 minutes and to release the oligonucleotide from the nucleic acid-labeled lectin, thereby recovering the supernatant after centrifugation. Subsequently, a PCR reaction was carried out using the recovered supernatant as a template, the PCR product was sequenced using a next generation sequencer (product name: “MiSeq”, Illumina, Inc.), and the number of reads of the detected base sequence was totaled.

In addition, a cDNA library was prepared from the total RNA of a single cell precipitated after centrifugation by using GenNext® RamDA-seq™ Single Cell Kit (Toyobo Co., Ltd.).

Subsequently, the sample was treated using Nextera XT DNA Sample Preparation Kit (Illumina, Inc.) and then sequenced using a next generation sequencer (product name: “NovaSeq 6000”, Illumina, Inc.), and the quality of the raw data was checked using FastQC v0.11.7 (https://www.bioinformatics.babraham.ac.uk/projects/fastqc/).

Then, trimming was carried out with Trimmomatic 0.38 (http://www.usadellab.org/cms/?page=trimmomatic), and mapping to the genome was carried out using HISAT2 v2.1.0 (http://daehwankimlab.github.io/hisat2/) and Bowtie2 2.3.5.1 (https://sourceforge.net/projects/bowtie-bio/files/bowtie2/2.3.5.1/). Further, mapping to the transcript was carried out using StringTie v1.3.4d (https://ccb.jhu.edu/software/stringtie/).

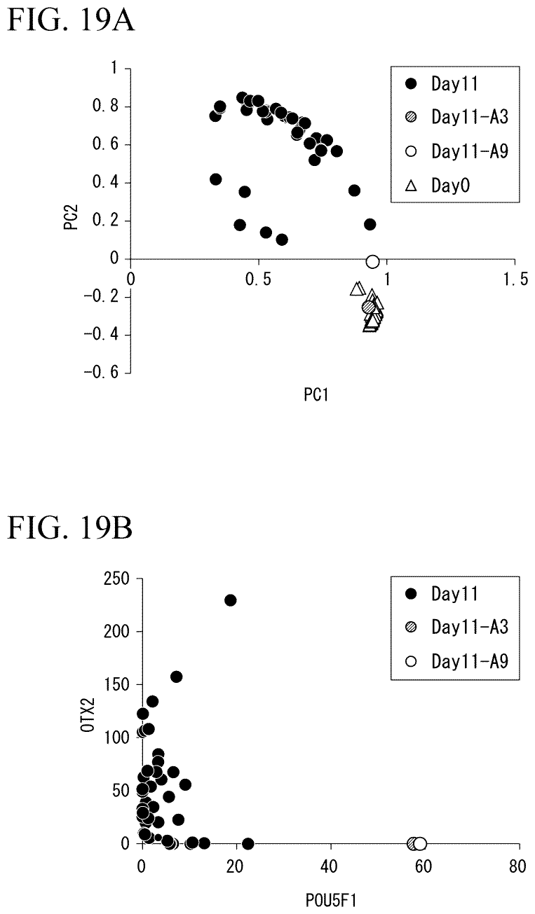

FIG. 19A is a graph showing the results obtained by subjecting the obtained glycan profile to main component analysis. As a result, it was revealed that the heterogeneity of the glycan profile is increased in the cell on the 11th day after differentiation induction as compared with the cell on the 0th day.

Subsequently, the expression of the neural differentiation marker OTX2 gene and the undifferentiated marker POU5F1 gene was checked in each of the cells of Day11-A3 and Day11-A9, which showed a glycan profile close to that on the 0th day.

FIG. 19B is a graph showing the results obtained by analyzing the expression level of each marker gene. As a result, it was revealed that the expression of the neural differentiation marker is low and the expression of the undifferentiation marker is high in each of the cells of Day11-A3 and Day11-A9. That is, it was revealed that these cells are in an undifferentiated state without being induced to differentiate even on the 11th day after differentiation induction to ectoderm (nerve).

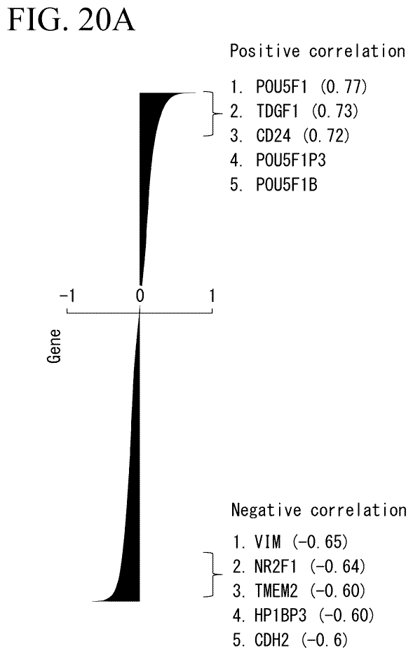

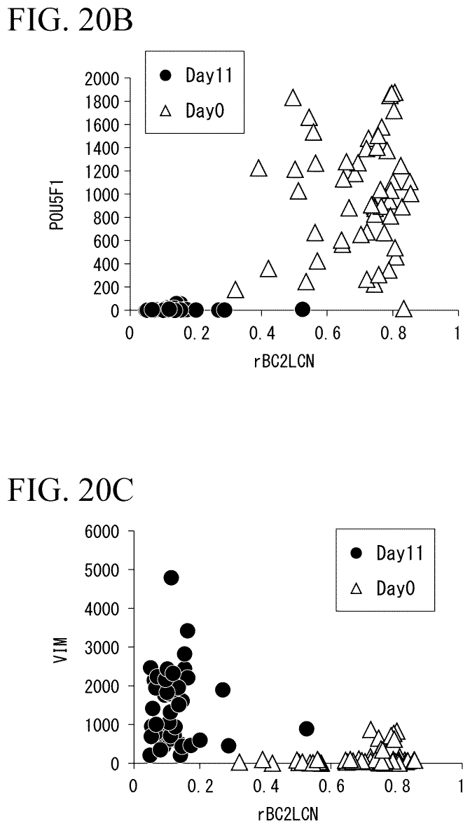

FIG. 20A is a graph showing the results of calculating the correlation coefficient between the amount of the rBC2LCN lectin bound to each cell, where the rBC2LCN lectin specifically binds to the human iPS cell, and the expression level of a group of 27,686 genes, and arranging them in descending order of numerical values.

As a result, it was revealed that the gene showing the highest positive correlation is the undifferentiation marker POU5F1. On the other hand, it was revealed that the gene showing the highest negative correlation is the neural differentiation marker VIM.

FIG. 20B is a scatter plot showing the amount of the rBC2LCN lectin bound to each cell and the expression level of the undifferentiation marker POU5F1 gene. FIG. 20C is a scatter plot showing the amount of the rBC2LCN lectin bound to each cell and the expression level of the neural differentiation marker VIM gene.

As a result, it was revealed that the binding amount of the rBC2LCN lectin shows a positive correlation with the expression level of the POU5F1 gene and shows a negative correlation with the expression level of the VIM gene. That is, it was revealed that the rBC2LCN lectin binds to a cell in which the expression of the undifferentiation marker POU5F1 gene is high and the expression of the neural differentiation marker VIM gene is low.

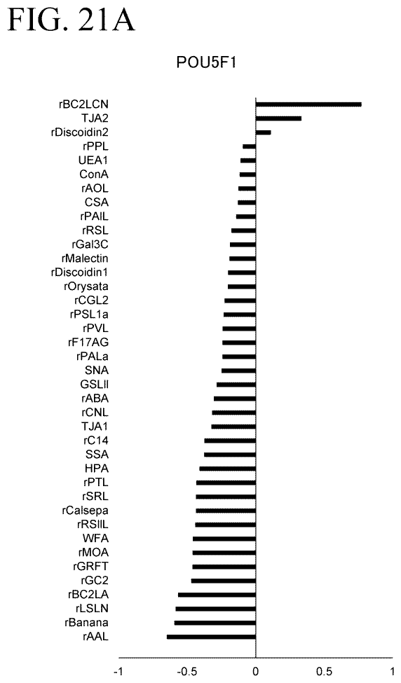

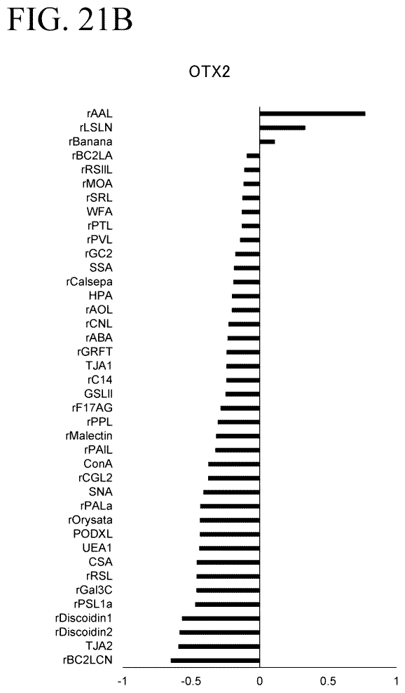

FIG. 21A is a graph in which the correlation coefficient between the expression level of the undifferentiation marker POU5F1 gene in each cell and the binding amount of 39 kinds of lectins is calculated and arranged in descending order. In addition, FIG. 21B is a graph in which the correlation coefficient between the expression level of the neural differentiation marker OTX2 gene in each cell and the binding amount of 39 kinds of lectins is calculated and arranged in descending order.

As a result, it was revealed that the lectin showing the highest positive correlation coefficient with respect to the expression level of the undifferentiation marker POU5F1 gene is rBC2LCN. In addition, it was revealed that the lectin showing the highest positive correlation coefficient with respect to the expression level of the neural differentiation marker OTX2 gene is rAAL. That is, it was conceivable that rAAL may show significantly high reactivity to nerve cells after differentiation induction as compared with human iPS cells.

From the above results, it was shown that according to the method of the present experimental example, it is possible to simultaneously measure glycans and genes, which are expressed in heterogeneous and diverse cell populations at a single cell level, and further, it is possible to reveal the relationship between the expressed glycans and the expressed genes from the obtained data, at a single cell level.

### INDUSTRIAL APPLICABILITY

According to the present invention, it is possible to provide a novel technique for analyzing a glycan.

1. A kit for profiling cell surface glycans on a single intact living cell, comprising:
ten or more kinds of glycan-binding substances, each of the glycan-binding substances being labeled with a respective nucleic acid including an encoding base sequence and an adapter sequence, the encoding base sequence being configured to identify one of the ten or more kinds of glycan-binding substances, the adapter sequence being configured to amplify the encoding base sequence by PCR; and
a buffer solution configured to serve as a solvent for the ten or more kinds of glycan-binding substances.

2. The kit according to claim 1, wherein the buffer solution is configured to maintain the ten or more kinds of glycan-binding substances as a mixture in a soluble state that allows simultaneous contact of the mixture with cell surface glycans on subject cells, subsequent physical removal of an unbound portion of the mixture from the buffer solution, and subsequent isolation of a single cell with a bound portion of the mixture while the single cell remains in an intact and living state.

3. The kit according to claim 2, wherein the buffer solution contains albumin.

4. The kit according to claim 3, wherein a concentration of the albumin in the buffer solution is between about 0.1% and 10% by mass.

5. The kit according to claim 1, wherein the ten or more kinds of glycan-binding substances comprise a group of recombinant lectins that have not been subjected to glycan modification.

6. The kit according to claim 1, further comprising primers, each of the primers including a sequence complementary to the adapter sequence of the nucleic acids associated with the ten or more kinds of glycan-binding substances,
wherein each of the primers is configured to hybridize to the adapter sequence to amplify the encoding base sequence by the PCR.

7. The kit according to claim 1, wherein each of the ten or more kinds of glycan-binding substances is bound to the respective nucleic acid via a spacer containing a group that is cleavable.

8. The kit according to claim 7, wherein the spacer contains a cleavable group that is cleavable upon irradiation with light.

9. The kit according to claim 6, wherein each of the ten or more kinds of glycan-binding substances is bound to the respective nucleic acid via a spacer containing a group that is cleavable.

10. The kit according to claim 6, further comprising instructions for analyzing glycans on a surface of subject cells, the instructions detailing:
bringing the mixture into contact with the subject cells in the buffer solution, resulting in a bound portion of the mixture binding to the glycans on the subject cells while an unbound portion of the mixture remains unbound in the buffer solution;
removing the unbound portion of the mixture from the buffer solution;
isolating a single cell from the subject cells after removing the unbound portion, the subject cells and the single cell being alive during isolation;
amplifying nucleic acids labeled on the glycan-binding substances that are associated with the single cell by the PCR using the adapter sequence of the nucleic acids and the primers; and
detecting the amplified nucleic acids, wherein the detected amplified nucleic acids and their quantities correspond to kinds and quantities of the glycans on the surface of the single cell.

11. The kit according to claim 1, wherein the ten or more kinds of glycan-binding substances recognize at least sialic acid (Sia), N-acetyl-glucosamine (GlcNAc), mannose (Man), D-galactose (Gal), N-acetyl-galactosamine (GalNAc), and L-fucose (Fuc), which are monosaccharides included in the glycans on the surface of subject cells.

12. The kit according to claim 11, wherein the ten or more kinds of glycan-binding substances further recognize D-glucose (Glc) and N-acetyl-lactosamine (LacNAc).

13. A kit for profiling cell surface glycans on a single cell, comprising:
a mixture of ten or more kinds of glycan-binding substances, each of the glycan-binding substances being bound to a respective nucleic acid;
wherein the respective nucleic acid comprises:
an encoding base sequence configured to identify the glycan-binding substance; and
at least one adapter sequence configured to amplify the encoding base sequence by PCR.

14. The kit according to claim 13, wherein the respective nucleic acid is bound to the glycan-binding substance via a spacer, and
wherein the spacer comprises a cleavable group configured to release the respective nucleic acid from the glycan-binding substance upon cleavage.

15. The kit according to claim 14, wherein the cleavable group is configured to be cleaved upon irradiation with light.

16. The kit according to claim 14, wherein the spacer comprises a polyethylene glycol chain, a polyacrylamide chain, a polyester chain, a polyurethane chain, or a copolymer thereof.

17. The kit according to claim 13, further comprising a buffer solution configured to serve as a solvent for the mixture of the ten or more kinds of glycan-binding substances, wherein the buffer solution comprises albumin at a concentration between about 0.1% and 10% by mass.

18. The kit according to claim 17, wherein the buffer solution comprises phosphate-buffered saline having a pH of about 7.4.

19. The kit according to claim 13, wherein the ten or more kinds of glycan-binding substances comprise a group of recombinant lectins, and wherein the kit further comprises primers configured to hybridize to the at least one adapter sequence to amplify the encoding base sequence by the PCR.

20. The kit according to claim 17, wherein the respective nucleic acid is a single-stranded DNA fragment.
